(19) United States
(12) Patent Application Publication
(10) Pub. No.: US 2024/0043551 A1
(43) Pub. Date: Feb. 8, 2024

(54) ANTI-TNFR2 ANTIBODY AND APPLICATION THEREOF

(71) Applicant: ADLAI NORTYE BIOPHARMA CO., LTD., Hangzhou, Zhejiang (CN)

(72) Inventors: Yi ZHANG, Hangzhou, Zhejiang (CN); Yonglin CHEN, Hangzhou, Zhejiang (CN)

(73) Assignee: ADLAI NORTYE BIOPHARMA CO., LTD., Hangzhou, Zhejiang (CN)

(21) Appl. No.: 18/266,600

(22) PCT Filed: Dec. 10, 2021
(86) PCT No.: PCT/CN2021/136952

§ 371 Date: Jun. 12, 2023

(30) Foreign Application Priority Data
Dec. 11, 2020 (WO) PCT/CN2020/135594

(51) Int. Cl. C07K 16/28; C12N 15/63; A61P 35/00

(52) U.S. Cl. CPC C07K 16/2878; C12N 15/63; A61P 35/00; C07K 2317/565; C07K 2317/567; C07K 2317/24; C07K 2317/14

(57) ABSTRACT

Provided is an antibody or an antigen binding fragment thereof capable of specifically binding to Tumor necrosis factor receptor type 2, which comprises a VH, a VL or both, said VH comprises at least one HCDR, which comprises an amino acid sequence selected from the group consisting of the amino acid sequences as set forth in SEQ ID NO: 13, SEQ ID NO: 15 and SEQ ID NO: 17. The antibody or an antigen binding fragment thereof binds strongly and specifically to TNFR2.

### BACKGROUND OF THE INVENTION

Tumor necrosis factor (TNF) is a key regulator of the immune system that initiates and orchestrates inflammation. TNF exerts its effect through 2 receptors, TNFR1 (TNF receptor type 1; CD120a; p55/60) and TNFR2 (TNF receptor type 2; CD120b; p75/80). TNFR2 results in recruitment of the TNF receptor-associated factor (TRAF) 2 and stimulates the pro-survival nuclear factor (NF)-KB pathway, contributes to immune regulation and tissue regeneration. (Front. Immunol. 2018 9:1170)

The unique biology and expression pattern of TNFR2 make it an attractive drug target for cancer therapy. First, TNFR2 expresses on a subset of regulatory T cells (Tregs) and MDSCs that can activate the proliferation of these cells through nuclear factor kappa B (NF-κB) pathway. TNFR2⁺ Treg has been shown to be most suppressive among all Treg population in tumor. Secondly, TNFR2 is also abundantly expressed on the surface of many human tumors. TNFR2 blocking antibody is expected to relieve TNFR2⁺ tumor-infiltrating Tregs mediated immunosuppression and directly kill TNFR2-expressing tumors, one stone that kill two birds. (Trends in Molecular Medicine, November 2017, Vol. 23, No. 11), Lastly, mice lacking the Tnfr2 gene failed to progress to systemic autoimmunity but have shown improved immune responses to tumors due to the lack of TNFR2-expressing Tregs (Torrey et al., Sci. Signal. 10, eaaf 8608 (2017)).

Several TNFR2 targeting agents have been developed. And novel assays and antibodies for TNFR2 are needed for its potential as a therapeutic target.

### SUMMARY OF THE INVENTION

The present disclosure provides an antibody or an antigen binding fragment thereof having one or more of the following properties: 1) binds strongly and specifically to recombinant TNFR2 protein and cell surface TNFR2 with sub-nanomolar affinity, not to its close homolog TNFR1; 2) cross-reacts with cyno TNFR2 with similar affinity, but not with mouse or rat TNFR2; 3) partially competes with TNFα for binding to TNFR2 receptor and blocks TNF/TNFR2 signaling pathway and inhibits TNF induced hTNFR2 cell death; 4) lacks of agonist activity towards TNFR2; 5) significantly inhibits tumor growth; 6) has safety and/or stability and shows no impact on body weight; 7) enhances TNF-dependent cell killing activity towards tumor cell line; 8) demonstrates strong immunomodulatory activity and/or potent anti-tumor efficacy in vivo.

In addition, the present disclosure also provides a method for producing the antibody or an antigen binding fragment thereof as well as pharmaceutical uses of the antibody or an antigen binding fragment thereof in preventing, alleviating and/or treating tumor.

In one aspect, the present disclosure provides an antibody or an antigen binding fragment thereof capable of specifically binding to Tumor necrosis factor receptor type 2 (TNFR2), which comprises a heavy chain variable region (VH), a light chain variable region (VL) or both, wherein said VH comprises at least one heavy chain complementarity determining region (HCDR), which comprises an amino acid sequence selected from the group consisting of the amino acid sequences as set forth in SEQ ID NO: 13, SEQ ID NO: 15 and SEQ ID NO: 17, and wherein the VL comprises at least one light chain complementarity determining region (LCDR), which comprises an amino acid sequence selected from the group consisting of the amino acid sequences as set forth in SEQ ID NO: 25, SEQ ID NO: 27 and SEQ ID NO: 29.

In some embodiments, said VH comprises an HCDR3, which comprises the amino acid sequence of SEQ ID NO: 17.

In some embodiments, said VH comprises an HCDR2, which comprises the amino acid sequence of SEQ ID NO: 15.

In some embodiments, said VH comprises HCDR1, which comprises the amino acid sequence of SEQ ID NO: 13.

In some embodiments, said VH comprises HCDR1 and HCDR3, which comprise the amino acid sequences of SEQ ID NO: 13 and SEQ ID NO: 17 respectively.

In some embodiments, said VH comprises HCDR2 and HCDR3, which comprise the amino acid sequences of SEQ ID NO: 15 and SEQ ID NO: 17 respectively.

In some embodiments, said VH comprises HCDR1 and HCDR2, which comprise the amino acid sequences of SEQ ID NO: 13 and SEQ ID NO: 15 respectively.

In some embodiments, said VH comprises HCDR1, HCDR2 and HCDR3, which comprise the amino acid sequences of SEQ ID NO: 13, SEQ ID NO: 15 and SEQ ID NO: 17 respectively.

In some embodiments, said VH comprises HFR1, said HFR1 comprises the amino acid sequence of SEQ ID NO: 35.

In some embodiments, said VH comprises HFR1, which comprises the amino acid sequence as set forth in SEQ ID NO: 42 (Formula (I)), wherein X1 is selected from hydrophobic amino acids and/or aliphatic amino acids.

In some embodiments, said HFR1 is selected from the group consisting of the amino acid sequences as set forth in SEQ ID NO: 35, SEQ ID NO: 22 and SEQ ID NO: 12.

In some embodiments, said VH comprises HFR2, which comprises the amino acid sequence as set forth in SEQ ID NO: 36 or SEQ ID NO: 14.

In some embodiments, said VH comprises HFR3, which comprises the amino acid sequence of SEQ ID NO: 37.

In some embodiments, said VH comprises HFR3, which comprises the amino acid sequence as set forth in SEQ ID NO: 43 (Formula (II)), wherein the X2 is selected from hydrophilic amino acids, wherein the X3 is selected from hydrophobic amino acids and/or aliphatic amino acids, and wherein the X4 is selected from hydrophobic amino acids and/or aromatic amino acids.

In some embodiments, said HFR3 is selected from the group consisting of the amino acid sequences as set forth in SEQ ID NO: 37, SEQ ID NO: 20, SEQ ID NO: 19 and SEQ ID NO: 16.

In some embodiments, said VH comprises HFR4, which comprises the amino acid sequences as set forth in SEQ ID NO: 44 (Formula (III)), wherein the X5 is selected from any amino acids.

In some embodiments, said HFR4 selected from the group consisting of the amino acid sequences as set forth in SEQ ID NO: 21, SEQ ID NO: 18 or SEQ ID NO: 23.

In some embodiments, said HFR comprises HFR1 comprising the amino acid sequence of SEQ ID NO: 12, said HFR comprises HFR2 comprising the amino acid sequence of SEQ ID NO: 14, said HFR comprises HFR3 comprising the amino acid sequence of SEQ ID NO: 16, and said HFR comprises HFR4 comprising the amino acid sequence of SEQ ID NO: 18.

In some embodiments, said HFR comprises HFR1 comprising the amino acid sequence of SEQ ID NO: 12, said HFR comprises HFR2 comprising the amino acid sequence of SEQ ID NO: 14, said HFR comprises HFR3 comprising the amino acid sequence of SEQ ID NO: 19, and said HFR comprises HFR4 comprising the amino acid sequence of SEQ ID NO: 18.

In some embodiments, said HFR comprises HFR1 comprising the amino acid sequence of SEQ ID NO: 12, said HFR comprises HFR2 comprising the amino acid sequence of SEQ ID NO: 14, said HFR comprises HFR3 comprising the amino acid sequence of SEQ ID NO: 20, and said HFR comprises HFR4 comprising the amino acid sequence of SEQ ID NO: 21.

In some embodiments, said HFR comprises HFR1 comprising the amino acid sequence of SEQ ID NO: 22, said HFR comprises HFR2 comprising the amino acid sequence of SEQ ID NO: 14, said HFR comprises HFR3 comprising the amino acid sequence of SEQ ID NO: 16, and said HFR comprises HFR4 comprising the amino acid sequence of SEQ ID NO: 23.

In some embodiments, said HFR comprises HFR1 comprising the amino acid sequence of SEQ ID NO: 22, said HFR comprises HFR2 comprising the amino acid sequence of SEQ ID NO: 14, said HFR comprises HFR3 comprising the amino acid sequence of SEQ ID NO: 16, and said HFR comprises HFR4 comprising the amino acid sequence of SEQ ID NO: 21.

In some embodiments, said VH comprises an amino acid sequence selected from the group consisting of the amino acid sequences as set forth in SEQ ID NO: 1, SEQ ID NO: 3, SEQ ID NO: 4, SEQ ID NO: 5, SEQ ID NO: 6 and SEQ ID NO: 7.

In some embodiments, said VL comprises LFR1, which comprise the amino acid sequences of SEQ ID NO: 38.

In some embodiments, said VL comprises LFR1, which comprises the amino acid sequences as set forth in SEQ ID NO: 45 (Formula (IV)), wherein the X6 is selected from hydrophobic amino acids and/or aliphatic amino acids, and wherein the X7 is selected from any amino acids.

In some embodiments, said VL comprises LFR2, which comprise the amino acid sequences of SEQ ID NO: 39.

In some embodiments, said VL comprises LFR2, which comprises the amino acid sequences as set forth in SEQ ID NO: 46 (Formula (V)), wherein the X8 is selected from small amino acid.

In some embodiments, said VL comprises LFR3, which comprises the amino acid sequences of SEQ ID NO: 40.

In some embodiments, said VL comprises LFR3, which comprises the amino acid sequences as set forth in SEQ ID NO: 47 (Formula (VI)), wherein the X9 is selected from negatively-charged amino acids or hydrophilic amino acids.

In some embodiments, said VL comprises LFR4, which comprises the amino acid sequences of SEQ ID NO: 41 or SEQ ID NO: 30.

In some embodiments, said VL comprises LCDR3, which comprises the amino acid sequence of SEQ ID NO: 29.

In some embodiments, said VL comprises LCDR2, which comprises the amino acid sequence of SEQ ID NO: 27.

In some embodiments, said VL comprises LCDR1, which comprises the amino acid sequence of SEQ ID NO: 25.

In some embodiments, said VL comprises LCDR1 and LCDR3, which comprise the amino acid sequences of SEQ ID NO: 25 and SEQ ID NO: 29 respectively.

In some embodiments, said VL comprises LCDR2 and LCDR3, which comprise the amino acid sequences of SEQ ID NO: 27 and SEQ ID NO: 29 respectively.

In some embodiments, said VL comprises LCDR1 and LCDR2, which comprise the amino acid sequences of SEQ ID NO: 25 and SEQ ID NO: 27 respectively.

In some embodiments, said VL comprises LCDR1, LCDR2 and LCDR3, which comprise the amino acid sequences of SEQ ID NO: 25, SEQ ID NO: 27 and SEQ ID NO: 29 respectively.

In some embodiments, said LFR comprises LFR1 selected from the group consisting of the amino acid sequences as set forth in SEQ ID NO: 24, said LFR comprises LFR2 selected from the group consisting of the amino acid sequences as set forth in SEQ ID NO: 26, said LFR comprises LFR3 selected from the group consisting of the amino acid sequences as set forth in SEQ ID NO: 28, and said LFR comprises LFR4 selected from the group consisting of the amino acid sequences as set forth in SEQ ID NO: 30.

In some embodiments, said LFR comprises LFR1, which comprises the amino acid sequence of SEQ ID NO: 24, said LFR comprises LFR2, which comprises the amino acid sequence of SEQ ID NO: 31, said LFR comprises LFR3, which comprises the amino acid sequence of SEQ ID NO: 28, and said LFR comprises LFR4, which comprises the amino acid sequence of SEQ ID NO: 30.

In some embodiments, said LFR comprises LFR1, which comprises the amino acid sequence of SEQ ID NO: 32, said LFR comprises LFR2, which comprises the amino acid sequence of SEQ ID NO: 26, said LFR comprises LFR3, which comprises the amino acid sequence of SEQ ID NO: 28, and said LFR comprises LFR4, which comprises the amino acid sequence of SEQ ID NO: 30.

In some embodiments, said LFR comprises LFR1, which comprises the amino acid sequence of SEQ ID NO: 33, said LFR comprises LFR2, which comprises the amino acid sequence of SEQ ID NO: 26, said LFR comprises LFR3, which comprises the amino acid sequence of SEQ ID NO: 34, and said LFR comprises LFR4, which comprises the amino acid sequence of SEQ ID NO: 30.

In some embodiments, said VL comprises an amino acid sequence selected from the group consisting of the amino acid sequences as set forth in SEQ ID NO: 8-11.

In some embodiments, said VH comprises HCDR3 comprising the amino acid sequences of SEQ ID NO: 17, and wherein said VL comprises LCDR3 comprising the amino acid sequences of SEQ ID NO: 29.

In some embodiments, said VH comprises HCDR1, HCDR2 and HCDR3, which comprise the amino acid sequences of SEQ ID NO: 13, SEQ ID NO: 15 and SEQ ID NO: 17 respectively, and wherein said VL comprises LCDR1, LCDR2 and LCDR3, which comprise the amino acid sequences of SEQ ID NO: 25, SEQ ID NO: 27 and SEQ ID NO: 29 respectively.

In some embodiments, said VH comprises an amino acid sequence selected from the group consisting of the amino acid sequences as set forth in SEQ ID NO: 1, SEQ ID NO: 3, SEQ ID NO: 4, SEQ ID NO: 5, SEQ ID NO: 6 and SEQ ID NO: 7, and wherein said VL comprises an amino acid sequence selected from the group consisting of the amino acid sequences as set forth in SEQ ID NO: 2, SEQ ID NO: 8, SEQ ID NO: 9, SEQ ID NO: 10 and SEQ ID NO: 11.

In some embodiments, said VH comprises an amino acid sequence of SEQ ID NO: 1, and wherein said VL comprises an amino acid sequence of SEQ ID NO: 2.

In some embodiments, said VH comprises an amino acid sequence of SEQ ID NO: 3, and wherein said VL comprises an amino acid sequence of SEQ ID NO: 8.

In some embodiments, said VH comprises an amino acid sequence of SEQ ID NO: 3, and wherein said VL comprises an amino acid sequence of SEQ ID NO: 9.

In some embodiments, said VH comprises an amino acid sequence of SEQ ID NO: 3, and wherein said VL comprises an amino acid sequence of SEQ ID NO: 10.

In some embodiments, said VH comprises an amino acid sequence of SEQ ID NO: 3, and wherein said VL comprises an amino acid sequence of SEQ ID NO: 11.

In some embodiments, said VH comprises an amino acid sequence of SEQ ID NO: 4, and wherein said VL comprises an amino acid sequence of SEQ ID NO: 8.

In some embodiments, said VH comprises an amino acid sequence of SEQ ID NO: 4, and wherein said VL comprises an amino acid sequence of SEQ ID NO: 9.

In some embodiments, said VH comprises an amino acid sequence of SEQ ID NO: 4, and wherein said VL comprises an amino acid sequence of SEQ ID NO: 10.

In some embodiments, said VH comprises an amino acid sequence of SEQ ID NO: 4, and wherein said VL comprises an amino acid sequence of SEQ ID NO: 11.

In some embodiments, said VH comprises an amino acid sequence of SEQ ID NO: 5, and wherein said VL comprises an amino acid sequence of SEQ ID NO: 8.

In some embodiments, said VH comprises an amino acid sequence of SEQ ID NO: 5, and wherein said VL comprises an amino acid sequence of SEQ ID NO: 9.

In some embodiments, said VH comprises an amino acid sequence of SEQ ID NO: 5, and wherein said VL comprises an amino acid sequence of SEQ ID NO: 10.

In some embodiments, said VH comprises an amino acid sequence of SEQ ID NO: 5, and wherein said VL comprises an amino acid sequence of SEQ ID NO: 11.

In some embodiments, said VH comprises an amino acid sequence of SEQ ID NO: 6, and wherein said VL comprises an amino acid sequence of SEQ ID NO: 8.

In some embodiments, said VH comprises an amino acid sequence of SEQ ID NO: 6, and wherein said VL comprises an amino acid sequence of SEQ ID NO: 9.

In some embodiments, said VH comprises an amino acid sequence of SEQ ID NO: 6, and wherein said VL comprises an amino acid sequence of SEQ ID NO: 10.

In some embodiments, said VH comprises an amino acid sequence of SEQ ID NO: 6, and wherein said VL comprises an amino acid sequence of SEQ ID NO: 11.

In some embodiments, said VH comprises an amino acid sequence of SEQ ID NO: 7, and wherein said VL comprises an amino acid sequence of SEQ ID NO: 8.

In some embodiments, said VH comprises an amino acid sequence of SEQ ID NO: 7, and wherein said VL comprises an amino acid sequence of SEQ ID NO: 9.

In some embodiments, said VH comprises an amino acid sequence of SEQ ID NO: 7, and wherein said VL comprises an amino acid sequence of SEQ ID NO: 10.

In some embodiments, said VH comprises an amino acid sequence of SEQ ID NO: 7, and wherein said VL comprises an amino acid sequence of SEQ ID NO: 11.

In some embodiments, said antibody is a rabbit antibody, a chimeric antibody or humanized antibody.

In some embodiments, said antibody or the antigen binding fragment thereof further comprises a heavy chain constant region of a human IgG and/or a light chain constant region of a human antibody.

In some embodiments, said antibody or the antigen binding fragment thereof comprises a human IgG1, IgG2, IgG3, or IgG4 heavy chain constant region.

In some embodiments, said antibody or the antigen binding fragment thereof comprises a human Kappa or Lambda light chain constant region.

In some embodiments, said antibody or the antigen binding fragment thereof is IgG, IgM, IgA, IgD or IgE.

In some embodiments, said antibody or the antigen binding fragment thereof is: a full-length antibody, or a scFv, Fv, sdFv, Fab, Fab′ or F(ab′)2.

In some embodiments, said TNFR2 is a human TNFR2 and/or a Cynomolgus TNFR2.

In another aspect, the present disclosure provided an isolated nucleic acid molecule, which encodes a heavy chain of the antibody or the antigen binding fragment thereof.

In another aspect, the present disclosure provided an isolated nucleic acid, which encodes a light chain of the antibody or the antigen binding fragment thereof.

In another aspect, the present disclosure provided an isolated nucleic acid, which encodes the antibody or the antigen binding fragment thereof.

In another aspect, the present disclosure provided an expression vector, which contains said nucleic acid.

In another aspect, the present disclosure provided a host cell, which contains said isolated nucleic acid and/or the expression vector.

In some embodiments, said host cell produces said antibody or the antigen binding fragment thereof.

In another aspect, the present disclosure provided a composition, which comprises said antibody or the antigen binding fragment thereof, and optionally a pharmaceutically acceptable carrier.

In another aspect, the present disclosure provided a method for preparing a TNFR2 antibody or the antigen binding fragment thereof, which comprises: culturing said host cell and recovering said antibody or said antigen binding fragment thereof.

In another aspect, the present disclosure provided a method for treating a tumor and/or for inhibiting the growth of tumor cells expressing TNFR2, which comprises: administering said antibody or an antigen binding fragment thereof, said nucleic acid, and/or said expression vector, said host cell, or said composition.

In another aspect, the present disclosure provided said antibody or an antigen binding fragment thereof, said nucleic acid, and/or said expression vector, said host cell, or said composition, for use in treating a tumor and/or for inhibiting the growth of tumor cells expressing TNFR2.

In another aspect, the present disclosure provided a use of said antibody or an antigen binding fragment thereof, said nucleic acid, and/or said expression vector, said host cell, or said composition in treating a tumor and/or for inhibiting the growth of tumor cells expressing TNFR2.

In another aspect, the present disclosure provided a use of said antibody or an antigen binding fragment thereof, said nucleic acid, and/or said expression vector, said host cell, or said composition in preparing a drug for treating a tumor and/or for inhibiting the growth of tumor cells expressing TNFR2.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWING

The novel features of the invention are set forth with particularity in the appended claims. A better understanding of the features and advantages of the present invention will be obtained by reference to the following detailed description that sets forth illustrative embodiments, in which the principles of the invention are employed, and the accompanying drawings (also “figure” and “FIG.” herein), of which:

FIG. 1 illustrates ELISA binding results of the antibodies of present disclosure.

FIG. 2A-2B illustrate ELISA binding results of the antibodies of present disclosure showing bind specificity to human TNFR2 not human TNFR1.

FIG. 3A-3C illustrate ELISA binding results of the antibodies of present disclosure showing cross reaction with cyno TNFR2 but not mouse nor rat TNFR2.

FIG. 4 illustrates the establishment of a human TNFR2 overexpressing Jurkat stable cell line.

FIG. 5A-5B illustrate FACS results of the antibodies of present disclosure binding to human TNFR2 on cell surface.

FIG. 6 illustrates the antibodies of present disclosure compete partially with TNFα for TNFR2 receptor binding.

FIG. 7A-7B illustrate the cell response to TNFα induced cell death.

FIG. 8 illustrates the antibodies of present disclosure inhibits TNFα induced human TNFR2 Jurkat cell death.

FIG. 9 illustrates the antibodies of present disclosure does not agonize TNFR2 receptor.

FIG. 10A-10B illustrate the expression pattern of TNFR1 and TNFR2 on Colo205 cell line.

FIG. 11A-11B illustrate the antibodies of present disclosure enhance TNF-dependent cell killing activity towards Colo205 cell line.

FIG. 12 illustrates in vivo PK profiles of the antibodies of present disclosure in mice.

FIG. 13 illustrates schematic diagram of the administration of the antibodies of present disclosure in mice.

FIG. 14A-14D illustrate the result of the antibodies of present disclosure in tumor inhibition in human TNFR2 mouse model.

FIG. 15A-15B illustrate the result of the antibodies of present disclosure in increasing tumor infiltrating CD4/8⁺ T cell.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The singular form “a,” “an” and “the,” as used herein, generally include plural references unless the context clearly dictates otherwise.

The term “antibody”, as used herein, generally refers to an immunoglobulin or an immunoglobulin-like molecule capable of specifically recognizing or binding to an antigen. An antibody may comprise a light chain (L) and a heavy chain (H). The light chains of an antibody can be classified as κ and λ light chains. The heavy chains can be classified as μ, δ, γ, α or ε, and the isotypes of an antibody are defined as IgM, IgD, IgG (e.g., IgG1, IgG2, IgG3 or IgG4 subtype), IgA and IgE, respectively. Each heavy chain may comprise a heavy chain variable region (VH) and a heavy chain constant region (CH). The heavy chain constant region may comprise three domains (CH1, CH2 and CH3). Each light chain may comprise a light chain variable region (VL) and a light chain constant region (CL). The light chain constant region may comprise a CL domain. The VH and VL regions can also be subdivided into regions with high variability known as complementarity determining regions (CDRs) interspersed with more conserved regions known as framework regions (FRs). Each VH and VL consists of 3 CDRs and 4 FRs arranged from N-terminal to C-terminal in the following order: FR1, CDR1, FR2, CDR2, FR3, CDR3, FR4. The variable regions (VH and VL) of each heavy/light chain pair form the antibody binding site, respectively. Distribution of amino acids to regions or domains follows the definition of Kabat Sequences of Proteins of Immunological Interest (National Institutes of Health, Bethesda, Md. (1987 and 1991)), or Chothia & Lesk (1987) J. Mol. Biol. 196:901-917; Chothia et al. (1989) Nature 342:878-883. The term “antibody” is not limited by any antibody-producing method. For example, it includes recombinant antibodies, monoclonal antibodies, and other forms of antibodies. In some cases, an antibody of the present disclosure is an isolated antibody.

The term “TNFR2”, as used herein, generally refers to Tumor necrosis factor receptor 2, also known as tumor necrosis factor receptor superfamily member 1B (TNFRSF1B) and CD120b, is a membrane receptor binding to tumor necrosis factor-alpha (TNFα). The human TNFR2 protein has NCBI login number NP_001057 while the mouse is NP_035740. Gene TNFRSF1A may encode the TNFR2 protein.

The term “tumor”, as used herein, generally refers to all types of cancer or neoplasm or malignant tumors found in mammals. The tumor may also comprise a tissue comprising malignant cells of the cancer. The tumor may comprise a solid tumor.

The term “hydrophobic amino acids”, as used herein, generally refers to amino acids having hydrophobic side chains. For example, said hydrophobic amino acids may comprise glycine (Gly), alanine (Ala), valine (Val), leucine (Leu), isoleucine (Ile), proline (Pro), phenylalanine (Phe), methionine (Met), and tryptophan (Trp). Said hydrophobic amino acids may tend to be repelled from water.

The term “aliphatic amino acid”, as used herein, generally refers to amino acids comprising an aliphatic side chain functional group. For example, said aliphatic amino acid may be non-polar and hydrophobic. For example, said aliphatic amino acid may comprise alanine, isoleucine, leucine, proline, and valine.

The term “small amino acids”, as used herein, generally refers to amino acids having relatively small volume. For example, said small amino acid may comprise alanine (Ala), Glycine (Gly), Serine (Ser), Asparagine (Asn), Aspartic acid (Asp), Cystine (Cys), Proline (Pro) and Threonine (Thr).

The term “negatively-charged amino acids”, as used herein, generally refers to amino acids being negative charged. For example, said negatively-charged amino acids may comprise Aspartic acid (Asp) and Glutamic acid (Glu).

The term “CDR” as used herein, generally refers to the complementarity determining region amino acid sequences of an antigen binding protein. These are the hypervariable regions of immunoglobulin heavy and light chains. There are three heavy chain and three light chain CDRs (or CDR regions) in the variable portion of an immunoglobulin. It will be apparent to those skilled in the art that there are various numbering systems for CDR sequences, e.g., Chothia (Chothia et al. (1989) Nature 342: 877-883), Kabat (Kabat et al., Sequences of Proteins of Immunological Interest, National Institutes of Health, Bethesda, Md. (1987 and 1991)). The Chothia numbering system is used for numbering the residues in an antibody of the present disclosure.

The term “antigen-binding fragment”, as used herein, generally refers to a portion of an immunoglobulin molecule. An antigen-binding fragment may comprise one light chain and part of a heavy chain with a single antigen-binding site. An antigen-binding fragment may be obtained by papain digestion of an immunoglobulin molecule. For example, an antigen-binding may be composed of one constant and one variable domain of each of the heavy and the light chain. The variable domain may contain the paratope (the antigen-binding site), comprising a set of the complementarity determining regions, at the amino-terminal end of the immunoglobulin molecule. The enzyme papain may be used to cleave an immunoglobulin molecule into two Fab fragments and one Fc fragment. The enzyme pepsin cleaves below the hinge region, so a F(ab′)2 fragment and a pFc′ fragment is formed. Divalent F(ab)2 or F(ab′)2 fragments have two antigen binding regions that are linked by disulfide bonds. Reduction of F(ab)2 or F(ab′)2 fragments produces 2 monovalent Fab or Fab′ fragments, which have a free sulfhydryl group that is useful for conjugation to other molecules.

The term “chimeric antibody”, as used herein, generally refers to an antibody in which the Variable (V) region of light and heavy chains is of mouse origin, while the Constant (C) region is of human origin. In general, the chimeric antibody may retain the specificity and affinity of the original mouse monoclonal antibody, but HAMA response may be significantly reduced.

The term “humanized antibody”, as used herein, generally refers to antibodies from non-human species whose protein sequences have been modified to increase their similarity to antibody variants produced naturally in humans. The amino acid sequence of a humanized antibody may be essentially identical to that of a human variant, despite the non-human origin of some of its complementarity determining region (CDR) segments responsible for the ability of the antibody to bind to its target antigen.

The term “fully human antibody”, as used herein, generally refers to an antibody with fully human amino acid sequence derived antibody region therapeutics where antigen specificity has been selected either in vivo by the use of genetically modified mice or by antibody engineering processes combined with screening.

The term “Fab fragment”, as used herein, generally refers to a portion (such as an antigen-binding fragment) of an immunoglobulin molecule. A Fab fragment may comprise one light chain and part of a heavy chain with a single antigen-binding site. A Fab fragment may be obtained by papain digestion of an immunoglobulin molecule. For example, a Fab fragment may be composed of one constant and one variable domain of each of the heavy and the light chain. The variable domain may contain the paratope (the antigen-binding site), comprising a set of the complementarity determining regions, at the amino-terminal end of the immunoglobulin molecule. The enzyme papain may be used to cleave an immunoglobulin molecule into two Fab fragments and one Fc fragment. The enzyme pepsin cleaves below the hinge region, so a F(ab′)2 fragment and a pFc′ fragment is formed. Divalent F(ab)2 or F(ab′)2 fragments have two antigen binding regions that are linked by disulfide bonds. Reduction of F(ab)2 or F(ab′)2 fragments produces 2 monovalent Fab or Fab′ fragments, which have a free sulfhydryl group that is useful for conjugation to other molecules.

The term “Fv fragment”, as used herein, generally refers to the smallest fragment made from enzymatic cleavage of IgG and IgM class antibodies. Fv fragments have the antigen-binding site made of the VH and VL regions, but they lack the CH1 and CL regions. The VH and VL chains may be held together in Fv fragments by non-covalent interactions.

The term “ScFv”, as used herein, generally refers to a single-chain antibody fragment. An ScFv may be a recombinant single chain polypeptide molecule in which light and heavy chain variable regions of an antibody are connected, either directly or via a peptide linker. Single chain antibodies (ScFv) generally do not include portions of the Fc region of antibody, although methods are known for adding such regions to known ScFv molecules if desired. See Helfrich et al., A rapid and versatile method for harnessing ScFv antibody fragments with various biological functions. J Immunol Methods 237: 131-145 (2000) and de Haard et al., Creating and engineering human antibodies for immunotherapy. Advanced Drug Delivery Reviews 31:5-31 (1998).

The term “isolated nucleic acid molecule or molecules” as used herein, generally refers to a polymeric form of nucleotides of any length, either deoxyribonucleotides or ribonucleotides, or analogs thereof, isolated from its native environment, or that is artificially synthesized.

The term “vector or vectors” as used herein, generally refers to a nucleic acid vehicle into which a polynucleotide encoding a protein can be inserted and expressed. The genetic material elements carried in the vector can be expressed in a host cell by transforming, transducing, or transfecting the host cell with the vector. Embodiments of vectors include: plasmids; phagemids; cosmid; artificial chromosomes. A vector may contain a variety of elements that control expression, including promoter sequences, transcriptional initiation sequences, enhancer sequences, selection elements, and reporter genes. In addition, the vector may also contain an origin of replication. It is also possible that the vector may include components that assist its entry into the cell, such as viral particles, liposomes or protein shells, but not only these substances.

The term “cell” as used herein, generally refers to a cell that may be used to carry the vector or vectors of the present disclosure, or be used to express or produce the antibody, the antigen binding fragment or variant of the present disclosure. A cell of the present disclosure may be a host cell. The cell may be a prokaryotic cell, a fungal cell, an insect cell, or another cell such as a CHO cell, or other cells suitable for antibody expression.

The term “conditions enabling expression”, as used herein, generally refers to conditions enabling the expression of the isolated antigen binding protein of the present disclosure. In some embodiments, such conditions may include but not limited to incubation time, temperature, and culture medium, and may depend on cell type and may be readily determined by a skilled arctician.

The term “about”, as used herein, generally refers to an approximation to a given value that would reasonably be inferred based on the ordinary skill in the art, including equivalents and approximations due to the experimental and/or measurement conditions for such given value. For example, it may refer to a value that is no more than 10% above or below the value being modified by the term.

In one aspect, the present disclosure provides an antibody or an antigen binding fragment thereof capable of specifically binding to Tumor necrosis factor receptor type 2 (TNFR2), which comprises a heavy chain variable region (VH), a light chain variable region (VL) or both, wherein said VH comprises at least one heavy chain complementarity determining region (HCDR), which comprises an amino acid sequence selected from the group consisting of the amino acid sequences as set forth in SEQ ID NO: 13, SEQ ID NO: 15 and SEQ ID NO: 17, and wherein the VL comprises at least one light chain complementarity determining region (LCDR), which comprises an amino acid sequence selected from the group consisting of the amino acid sequences as set forth in SEQ ID NO: 25, SEQ ID NO: 27 and SEQ ID NO: 29.

In the present disclosure, said VH may comprise an HCDR3, which may comprise the amino acid sequence of SEQ ID NO: 17.

In the present disclosure, said VH may comprise an HCDR2, which may comprise the amino acid sequence of SEQ ID NO: 15.

In the present disclosure, said VH may comprise HCDR1, which may comprise the amino acid sequence of SEQ ID NO: 13.

In the present disclosure, said VH may comprise HCDR1 and HCDR3, which may comprise the amino acid sequences of SEQ ID NO: 13 and SEQ ID NO: 17 respectively.

In the present disclosure, said VH may comprise HCDR2 and HCDR3, which may comprise the amino acid sequences of SEQ ID NO: 15 and SEQ ID NO: 17 respectively.

In the present disclosure, said VH may comprise HCDR1 and HCDR2, which may comprise the amino acid sequences of SEQ ID NO: 13 and SEQ ID NO: 15 respectively.

In the present disclosure, said VH may comprise HCDR1, HCDR2 and HCDR3, which may comprise the amino acid sequences of SEQ ID NO: 13, SEQ ID NO: 15 and SEQ ID NO: 17 respectively.

In the present disclosure, the sequence may be determined according to the IMGT definition.

In the present disclosure, said VH may comprise HFR1, said HFR1 may comprise the amino acid sequence of SEQ ID NO: 35.

In the present disclosure, said VH may comprise HFR1, which may comprise the amino acid sequence as set forth in SEQ ID NO: 42 (EVQLVESGGGL X₁ QPGGSLRLSCAAS), wherein X₁ is selected from hydrophobic amino acids and/or aliphatic amino acids. For example, X₁ is selected from V, I, L, F, W, Y and M.

In the present disclosure, said HFR1 may be selected from the group consisting of the amino acid sequences as set forth in SEQ ID NO: 35, SEQ ID NO: 22 and SEQ ID NO: 12. For example, said HFR1 may be selected from the group consisting of the amino acid sequences as set forth in SEQ ID NO: 22 and SEQ ID NO: 12.

In the present disclosure, said VH may comprise HFR2, which may comprise the amino acid sequence as set forth in SEQ ID NO: 36 or SEQ ID NO: 14.

In the present disclosure, said VH may comprise HFR3, which may comprise the amino acid sequence of SEQ ID NO: 37.

In the present disclosure, said VH may comprise HFR3, which may comprise the amino acid sequence as set forth in SEQ ID NO: 43 (YYSNWAKSRFTISRD X₂ SKNT X₃ YLQMNSLRAEDTAVY X₄C), wherein the X₂ may be selected from hydrophilic amino acids, wherein the X₃ may be selected from hydrophobic amino acids and/or aliphatic amino acids, and wherein the X₄ may be selected from hydrophobic amino acids and/or aromatic amino acids.

For example, X₂ may be selected from N, T, S, H, Q, E, D, K and R; For example, X₃ may be selected from V, L, I, F, W, Y and M; For example, X₄ may be selected from F, Y, H, V, I, L, W and M.

In the present disclosure, said HFR3 may be selected from the group consisting of the amino acid sequences as set forth in SEQ ID NO: 37, SEQ ID NO: 20, SEQ ID NO: 19 and SEQ ID NO: 16. For example, aid HFR3 may be selected from the group consisting of the amino acid sequences as set forth in SEQ ID NO: 16, SEQ ID NO: 20 and SEQ ID NO: 19.

In the present disclosure, said VH may comprise HFR4, which may comprise the amino acid sequences as set forth in SEQ ID NO: 44 (WG X₅GTLVTVSS), wherein the X₅ is selected from any amino acids.

For example, X₅ may be selected from P, Q and R.

In the present disclosure, said HFR4 may be selected from the group consisting of the amino acid sequences as set forth in SEQ ID NO: 21, SEQ ID NO: 18 or SEQ ID NO: 23. For example, said HFR4 may be selected from the group consisting of the amino acid sequences as set forth in SEQ ID NO: 21 and SEQ ID NO: 18.

In the present disclosure, said HFR may comprise HFR1 comprising the amino acid sequence of SEQ ID NO: 12, said HFR may comprise HFR2 comprising the amino acid sequence of SEQ ID NO: 14, said HFR may comprise HFR3 comprising the amino acid sequence of SEQ ID NO: 16, and said HFR may comprise HFR4 comprising the amino acid sequence of SEQ ID NO: 18.

In the present disclosure, said HFR may comprise HFR1 comprising the amino acid sequence of SEQ ID NO: 12, said HFR may comprise HFR2 comprising the amino acid sequence of SEQ ID NO: 14, said HFR may comprise HFR3 comprising the amino acid sequence of SEQ ID NO: 19, and said HFR may comprise HFR4 comprising the amino acid sequence of SEQ ID NO: 18.

In the present disclosure, said HFR may comprise HFR1 comprising the amino acid sequence of SEQ ID NO: 12, said HFR may comprise HFR2 comprising the amino acid sequence of SEQ ID NO: 14, said HFR may comprise HFR3 comprising the amino acid sequence of SEQ ID NO: 20, and said HFR may comprise HFR4 comprising the amino acid sequence of SEQ ID NO: 21.

In the present disclosure, said HFR may comprise HFR1 comprising the amino acid sequence of SEQ ID NO: 22, said HFR may comprise HFR2 comprising the amino acid sequence of SEQ ID NO: 14, said HFR may comprise HFR3 comprising the amino acid sequence of SEQ ID NO: 16, and said HFR may comprise HFR4 comprising the amino acid sequence of SEQ ID NO: 23.

In the present disclosure, said HFR may comprise HFR1 comprising the amino acid sequence of SEQ ID NO: 22, said HFR may comprise HFR2 comprising the amino acid sequence of SEQ ID NO: 14, said HFR may comprise HFR3 comprising the amino acid sequence of SEQ ID NO: 16, and said HFR may comprise HFR4 comprising the amino acid sequence of SEQ ID NO: 21.

In the present disclosure, said VH may comprise an amino acid sequence selected from the group consisting of the amino acid sequences as set forth in SEQ ID NO: 1, SEQ ID NO: 3, SEQ ID NO: 4, SEQ ID NO: 5, SEQ ID NO: 6 and SEQ ID NO: 7.

In the present disclosure, said VL may comprise LFR1, which comprise the amino acid sequences of SEQ ID NO: 38.

In the present disclosure, said VL may comprise LFR1, which may comprise the amino acid sequences as set forth in SEQ ID NO: 45 (DIQ X₆ TQSPS X₇LSASVGDRVTITCQVS), wherein the X₆ is selected from hydrophobic amino acids and/or aliphatic amino acids, and wherein the X₇ is selected from any amino acids.

For example, X₆ may be selected from L, M, V, I, F, W and Y. For example, X₇ may be selected from S and F.

In the present disclosure, said LFR1 may be selected from the group consisting of the amino acid sequences as set forth in SEQ ID NO: 38, SEQ ID NO: 24, SEQ ID NO: 32 and SEQ ID NO: 33. For example, said LFR1 may be selected from the group consisting of the amino acid sequences as set forth in SEQ ID NO: 24, SEQ ID NO: 32 and SEQ ID NO: 33.

In the present disclosure, said VL may comprise LFR2, which comprise the amino acid sequences of SEQ ID NO: 39.

In the present disclosure, said VL may comprise LFR2, which may comprise the amino acid sequences as set forth in SEQ ID NO: 46 (LSWYQQKPGK X₈ PKLLIY), wherein the X₈ is selected from small amino acid.

For example, X₈ may be selected from A, P, G and S.

In the present disclosure, said LFR2 may be selected from the group consisting of the amino acid sequences as set forth in SEQ ID NO: 31 and SEQ ID NO: 26.

In the present disclosure, said VL may comprise LFR3, which may comprise the amino acid sequences of SEQ ID NO: 40.

In the present disclosure, said VL may comprise LFR3, which may comprise the amino acid sequences as set forth in SEQ ID NO: 47 (TLASGVPSRFSGSGSGT X₉FTLTISSLQPEDFATYYC), wherein the X₉ is selected from negatively-charged amino acids or hydrophilic amino acids.

For example, X₉ may be selected from D, E, S, T, H, N, Q, K and R.

In the present disclosure, said LFR3 may be selected from the group consisting of the amino acid sequences as set forth in SEQ ID NO: 40, SEQ ID NO: 28 and SEQ ID NO: 34. For example, said LFR3 may be selected from the group consisting of the amino acid sequences as set forth in SEQ ID NO: 28 and SEQ ID NO: 34.

In the present disclosure, said VL may comprise LFR4, which may comprise the amino acid sequences of SEQ ID NO: 41 or SEQ ID NO: 30.

In the present disclosure, said VL may comprise LCDR3, which may comprise the amino acid sequence of SEQ ID NO: 29.

In the present disclosure, said VL may comprise LCDR2, which may comprise the amino acid sequence of SEQ ID NO: 27.

In the present disclosure, said VL may comprise LCDR1, which may comprise the amino acid sequence of SEQ ID NO: 25.

In the present disclosure, said VL may comprise LCDR1 and LCDR3, which comprise the amino acid sequences of SEQ ID NO: 25 and SEQ ID NO: 29 respectively.

In the present disclosure, said VL may comprise LCDR2 and LCDR3, which comprise the amino acid sequences of SEQ ID NO: 27 and SEQ ID NO: 29 respectively.

In the present disclosure, said VL may comprise LCDR1 and LCDR2, which comprise the amino acid sequences of SEQ ID NO: 25 and SEQ ID NO: 27 respectively.

In the present disclosure, said VL may comprise LCDR1, LCDR2 and LCDR3, which comprise the amino acid sequences of SEQ ID NO: 25, SEQ ID NO: 27 and SEQ ID NO: 29 respectively.

In the present disclosure, said LFR may comprise LFR1 selected from the group consisting of the amino acid sequences as set forth in SEQ ID NO: 24, said LFR may comprise LFR2 selected from the group consisting of the amino acid sequences as set forth in SEQ ID NO: 26, said LFR may comprise LFR3 selected from the group consisting of the amino acid sequences as set forth in SEQ ID NO: 28, and said LFR may comprise LFR4 selected from the group consisting of the amino acid sequences as set forth in SEQ ID NO: 30.

In the present disclosure, said LFR may comprise LFR1, which may comprise the amino acid sequence of SEQ ID NO: 24, said LFR may comprise LFR2, which may comprise the amino acid sequence of SEQ ID NO: 31, said LFR may comprise LFR3, which may comprise the amino acid sequence of SEQ ID NO: 28, and said LFR may comprise LFR4, which may comprise the amino acid sequence of SEQ ID NO: 30.

In the present disclosure, said LFR may comprise LFR1, which may comprise the amino acid sequence of SEQ ID NO: 32, said LFR may comprise LFR2, which may comprise the amino acid sequence of SEQ ID NO: 26, said LFR may comprise LFR3, which may comprise the amino acid sequence of SEQ ID NO: 28, and said LFR may comprise LFR4, which may comprise the amino acid sequence of SEQ ID NO: 30.

In the present disclosure, said LFR may comprise LFR1, which may comprise the amino acid sequence of SEQ ID NO: 33, said LFR may comprise LFR2, which may comprise the amino acid sequence of SEQ ID NO: 26, said LFR may comprise LFR3, which may comprise the amino acid sequence of SEQ ID NO: 34, and said LFR may comprise LFR4, which may comprise the amino acid sequence of SEQ ID NO: 30.

In the present disclosure, said VL may comprise an amino acid sequence selected from the group consisting of the amino acid sequences as set forth in SEQ ID NO: 8-11.

In the present disclosure, said VH may comprise HCDR3 comprising the amino acid sequences of SEQ ID NO: 17, and wherein said VL may comprise LCDR3 comprising the amino acid sequences of SEQ ID NO: 29.

In the present disclosure, said VH may comprise HCDR1, HCDR2 and HCDR3, which comprise the amino acid sequences of SEQ ID NO: 13, SEQ ID NO: 15 and SEQ ID NO: 17 respectively, and wherein said VL may comprise LCDR1, LCDR2 and LCDR3, which comprise the amino acid sequences of SEQ ID NO: 25, SEQ ID NO: 27 and SEQ ID NO: 29 respectively.

In the present disclosure, said VH may comprise an amino acid sequence selected from the group consisting of the amino acid sequences as set forth in SEQ ID NO: 1, SEQ ID NO: 3, SEQ ID NO: 4, SEQ ID NO: 5, SEQ ID NO: 6 and SEQ ID NO: 7, and wherein said VL may comprise an amino acid sequence selected from the group consisting of the amino acid sequences as set forth in SEQ ID NO: 2, SEQ ID NO: 8, SEQ ID NO: 9, SEQ ID NO: 10 and SEQ ID NO: 11.

In the present disclosure, said VH may comprise an amino acid sequence of SEQ ID NO: 1, and wherein said VL may comprise an amino acid sequence of SEQ ID NO: 2.

In the present disclosure, said VH may comprise an amino acid sequence of SEQ ID NO: 3, and wherein said VL may comprise an amino acid sequence of SEQ ID NO: 8.

In the present disclosure, said VH may comprise an amino acid sequence of SEQ ID NO: 3, and wherein said VL may comprise an amino acid sequence of SEQ ID NO: 9.

In the present disclosure, said VH may comprise an amino acid sequence of SEQ ID NO: 3, and wherein said VL may comprise an amino acid sequence of SEQ ID NO: 10.

In the present disclosure, said VH may comprise an amino acid sequence of SEQ ID NO: 3, and wherein said VL may comprise an amino acid sequence of SEQ ID NO: 11.

In the present disclosure, said VH may comprise an amino acid sequence of SEQ ID NO: 4, and wherein said VL may comprise an amino acid sequence of SEQ ID NO: 8.

In the present disclosure, said VH may comprise an amino acid sequence of SEQ ID NO: 4, and wherein said VL may comprise an amino acid sequence of SEQ ID NO: 9.

In the present disclosure, said VH may comprise an amino acid sequence of SEQ ID NO: 4, and wherein said VL may comprise an amino acid sequence of SEQ ID NO: 10.

In the present disclosure, said VH may comprise an amino acid sequence of SEQ ID NO: 4, and wherein said VL may comprise an amino acid sequence of SEQ ID NO: 11.

In the present disclosure, said VH may comprise an amino acid sequence of SEQ ID NO: 5, and wherein said VL may comprise an amino acid sequence of SEQ ID NO: 8.

In the present disclosure, said VH may comprise an amino acid sequence of SEQ ID NO: 5, and wherein said VL may comprise an amino acid sequence of SEQ ID NO: 9.

In the present disclosure, said VH may comprise an amino acid sequence of SEQ ID NO: 5, and wherein said VL may comprise an amino acid sequence of SEQ ID NO: 10.

In the present disclosure, said VH may comprise an amino acid sequence of SEQ ID NO: 5, and wherein said VL may comprise an amino acid sequence of SEQ ID NO: 11.

In the present disclosure, said VH may comprise an amino acid sequence of SEQ ID NO: 6, and wherein said VL may comprise an amino acid sequence of SEQ ID NO: 8.

In the present disclosure, said VH may comprise an amino acid sequence of SEQ ID NO: 6, and wherein said VL may comprise an amino acid sequence of SEQ ID NO: 9.

In the present disclosure, said VH may comprise an amino acid sequence of SEQ ID NO: 6, and wherein said VL may comprise an amino acid sequence of SEQ ID NO: 10.

In the present disclosure, said VH may comprise an amino acid sequence of SEQ ID NO: 6, and wherein said VL may comprise an amino acid sequence of SEQ ID NO: 11.

In the present disclosure, said VH may comprise an amino acid sequence of SEQ ID NO: 7, and wherein said VL may comprise an amino acid sequence of SEQ ID NO: 8.

In the present disclosure, said VH may comprise an amino acid sequence of SEQ ID NO: 7, and wherein said VL may comprise an amino acid sequence of SEQ ID NO: 9.

In the present disclosure, said VH may comprise an amino acid sequence of SEQ ID NO: 7, and wherein said VL may comprise an amino acid sequence of SEQ ID NO: 10.

In the present disclosure, said VH may comprise an amino acid sequence of SEQ ID NO: 7, and wherein said VL may comprise an amino acid sequence of SEQ ID NO: 11.

For example, the VH may comprise HCDR1-3 and H-FR1-4, the HCDR1 may comprise an amino acid sequence as set forth in SEQ ID NO. 13, the HCDR2 may comprise an amino acid sequence as set forth in SEQ ID NO. 15, and the HCDR3 may comprise an amino acid sequence as set forth in SEQ ID NO. 17; the H-FR1 may comprises an amino acid sequence as set forth in SEQ ID NO.12, the H-FR2 may comprises an amino acid sequence as set forth in SEQ ID NO.14, the H-FR3 may comprises an amino acid sequence as set forth in SEQ ID NO.16, and the H-FR4 may comprises an amino acid sequence as set forth in SEQ ID NO.18; the VH may comprise an amino sequence as set forth in SEQ ID NO.3; the heavy chain constant region may comprise an amino acid sequence as set forth in SEQ ID NO. 48. And the VL may comprise LCDR1-3 and L-FR1-4, the LCDR1 may comprise an amino acid sequence as set forth in SEQ ID NO. 25, the LCDR2 may comprise an amino acid sequence as set forth in SEQ ID NO. 27, and the LCDR3 may comprise an sequence as set forth in SEQ ID NO.24, the L-FR2 may comprises an amino acid sequence as set forth in SEQ ID NO.26, the L-FR3 may comprises an amino acid sequence as set forth in SEQ ID NO.28 and the L-FR4 may comprises an amino acid sequence as set forth in SEQ ID NO.30; the VL may comprise an amino acid sequence as set forth in SEQ ID NO.8. And the antibody comprises the above VH and VL may be TN36-2.

For example, the VH may comprise HCDR1-3 and H-FR1-4, the HCDR1 may comprise an amino acid sequence as set forth in SEQ ID NO. 13, the HCDR2 may comprise an amino acid sequence as set forth in SEQ ID NO. 15, and the HCDR3 may comprise an amino acid sequence as set forth in SEQ ID NO. 17; the H-FR1 may comprises an amino acid sequence as set forth in SEQ ID NO.12, the H-FR2 may comprises an amino acid sequence as set forth in SEQ ID NO.14, the H-FR3 may comprises an amino acid sequence as set forth in SEQ ID NO.16, and the H-FR4 may comprises an amino acid sequence as set forth in SEQ ID NO.18; the VH may comprise an amino sequence as set forth in SEQ ID NO.3; the heavy chain constant region may comprise an amino acid sequence as set forth in SEQ ID NO. 48. And the VL may comprise LCDR1-3 and L-FR1-4, the LCDR1 may comprise an amino acid sequence as set forth in SEQ ID NO. 25, the LCDR2 may comprise an amino acid sequence as set forth in SEQ ID NO. 27, and the LCDR3 may comprise an amino acid sequence as set forth in SEQ ID NO. 29; the L-FR1 may comprises an amino acid sequence as set forth in SEQ ID NO.24, the L-FR2 may comprises an amino acid sequence as set forth in SEQ ID NO.31, the L-FR3 may comprises an amino acid sequence as set forth in SEQ ID NO.28 and the L-FR4 may comprises an amino acid sequence as set forth in SEQ ID NO.30; the VL may comprise an amino acid sequence as set forth in SEQ ID NO.9. And the antibody comprises the above VH and VL may be TN36-3.

For example, the VH may comprise HCDR1-3 and H-FR1-4, the HCDR1 may comprise an amino acid sequence as set forth in SEQ ID NO. 13, the HCDR2 may comprise an amino acid sequence as set forth in SEQ ID NO. 15, and the HCDR3 may comprise an amino acid sequence as set forth in SEQ ID NO. 17; the H-FR1 may comprises an amino acid sequence as set forth in SEQ ID NO.12, the H-FR2 may comprises an amino acid sequence as set forth in SEQ ID NO.14, the H-FR3 may comprises an amino acid sequence as set forth in SEQ ID NO.16, and the H-FR4 may comprises an amino acid sequence as set forth in SEQ ID NO.18; the VH may comprise an amino sequence as set forth in SEQ ID NO.3; the heavy chain constant region may comprise an amino acid sequence as set forth in SEQ ID NO. 48. And the VL may comprise LCDR1-3 and L-FR1-4, the LCDR1 may comprise an amino acid sequence as set forth in SEQ ID NO. 25, the LCDR2 may comprise an amino acid sequence as set forth in SEQ ID NO. 27, and the LCDR3 may comprise an amino acid sequence as set forth in SEQ ID NO. 29; the L-FR1 may comprises an amino acid sequence as set forth in SEQ ID NO.32, the L-FR2 may comprises an amino acid sequence as set forth in SEQ ID NO.26, the L-FR3 may comprises an amino acid sequence as set forth in SEQ ID NO.28 and the L-FR4 may comprises an amino acid sequence as set forth in SEQ ID NO.30; the VL may comprise an amino acid sequence as set forth in SEQ ID NO.10. And the antibody comprises the above VH and VL may be TN36-4.

For example, the VH may comprise HCDR1-3 and H-FR1-4, the HCDR1 may comprise an amino acid sequence as set forth in SEQ ID NO. 13, the HCDR2 may comprise an amino acid sequence as set forth in SEQ ID NO. 15, and the HCDR3 may comprise an amino acid sequence as set forth in SEQ ID NO. 17; the H-FR1 may comprises an amino acid sequence as set forth in SEQ ID NO.12, the H-FR2 may comprises an amino acid sequence as set forth in SEQ ID NO.14, the H-FR3 may comprises an amino acid sequence as set forth in SEQ ID NO.16, and the H-FR4 may comprises an amino acid sequence as set forth in SEQ ID NO.18; the VH may comprise an amino sequence as set forth in SEQ ID NO.3; the heavy chain constant region may comprise an amino acid sequence as set forth in SEQ ID NO. 48. And the VL may comprise LCDR1-3 and L-FR1-4, the LCDR1 may comprise an amino acid sequence as set forth in SEQ ID NO. 25, the LCDR2 may comprise an amino acid sequence as set forth in SEQ ID NO. 27, and the LCDR3 may comprise an amino acid sequence as set forth in SEQ ID NO. 29; the L-FR1 may comprises an amino acid sequence as set forth in SEQ ID NO.33, the L-FR2 may comprises an amino acid sequence as set forth in SEQ ID NO.26, the L-FR3 may comprises an amino acid sequence as set forth in SEQ ID NO.34 and the L-FR4 may comprises an amino acid sequence as set forth in SEQ ID NO.30; the VL may comprise an amino acid sequence as set forth in SEQ ID NO.11. And the antibody comprises the above VH and VL may be TN36-5.

For example, the VH may comprise HCDR1-3 and H-FR1-4, the HCDR1 may comprise an amino acid sequence as set forth in SEQ ID NO. 13, the HCDR2 may comprise an amino acid sequence as set forth in SEQ ID NO. 15, and the HCDR3 may comprise an amino acid sequence as set forth in SEQ ID NO. 17; the H-FR1 may comprises an amino acid sequence as set forth in SEQ ID NO.12, the H-FR2 may comprises an amino acid sequence as set forth in SEQ ID NO.14, the H-FR3 may comprises an amino acid sequence as set forth in SEQ ID NO.19, and the H-FR4 may comprises an amino acid sequence as set forth in SEQ ID NO.18; the VH may comprise an amino sequence as set forth in SEQ ID NO.4; the heavy chain constant region may comprise an amino acid sequence as set forth in SEQ ID NO. 48. And the VL may comprise LCDR1-3 and L-FR1-4, the LCDR1 may comprise an amino acid sequence as set forth in SEQ ID NO. 25, the LCDR2 may comprise an amino acid sequence as set forth in SEQ ID NO. 27, and the LCDR3 may comprise an amino acid sequence as set forth in SEQ ID NO. 29; the L-FR1 may comprises an amino acid sequence as set forth in SEQ ID NO.24, the L-FR2 may comprises an amino acid sequence as set forth in SEQ ID NO.26, the L-FR3 may comprises an amino acid sequence as set forth in SEQ ID NO.28 and the L-FR4 may comprises an amino acid sequence as set forth in SEQ ID NO.30; the VL may comprise an amino acid sequence as set forth in SEQ ID NO.8. And the antibody comprises the above VH and VL may be TN36-6.

For example, the VH may comprise HCDR1-3 and H-FR1-4, the HCDR1 may comprise an amino acid sequence as set forth in SEQ ID NO. 13, the HCDR2 may comprise an amino acid sequence as set forth in SEQ ID NO. 15, and the HCDR3 may comprise an amino acid sequence as set forth in SEQ ID NO. 17; the H-FR1 may comprises an amino acid sequence as set forth in SEQ ID NO.12, the H-FR2 may comprises an amino acid sequence as set forth in SEQ ID NO.14, the H-FR3 may comprises an amino acid sequence as set forth in SEQ ID NO.19, and the H-FR4 may comprises an amino acid sequence as set forth in SEQ ID NO.18; the VH may comprise an amino sequence as set forth in SEQ ID NO.4; the heavy chain constant region may comprise an amino acid sequence as set forth in SEQ ID NO. 48. And the VL may comprise LCDR1-3 and L-FR1-4, the LCDR1 may comprise an amino acid sequence as set forth in SEQ ID NO. 25, the LCDR2 may comprise an amino acid sequence as set forth in SEQ ID NO. 27, and the LCDR3 may comprise an amino acid sequence as set forth in SEQ ID NO. 29; the L-FR1 may comprises an amino acid sequence as set forth in SEQ ID NO.24, the L-FR2 may comprises an amino acid sequence as set forth in SEQ ID NO.31, the L-FR3 may comprises an amino acid sequence as set forth in SEQ ID NO.28 and the L-FR4 may comprises an amino acid sequence as set forth in SEQ ID NO.30; the VL may comprise an amino acid sequence as set forth in SEQ ID NO.9. And the antibody comprises the above VH and VL may be TN36-7.

For example, the VH may comprise HCDR1-3 and H-FR1-4, the HCDR1 may comprise an amino acid sequence as set forth in SEQ ID NO. 13, the HCDR2 may comprise an amino acid sequence as set forth in SEQ ID NO. 15, and the HCDR3 may comprise an amino acid sequence as set forth in SEQ ID NO. 17; the H-FR1 may comprises an amino acid sequence as set forth in SEQ ID NO.12, the H-FR2 may comprises an amino acid sequence as set forth in SEQ ID NO.14, the H-FR3 may comprises an amino acid sequence as set forth in SEQ ID NO.19, and the H-FR4 may comprises an amino acid sequence as set forth in SEQ ID NO.18; the VH may comprise an amino sequence as set forth in SEQ ID NO.4; the heavy chain constant region may comprise an amino acid sequence as set forth in SEQ ID NO. 48. And the VL may comprise LCDR1-3 and L-FR1-4, the LCDR1 may comprise an amino acid sequence as set forth in SEQ ID NO. 25, the LCDR2 may comprise an amino acid sequence as set forth in SEQ ID NO. 27, and the LCDR3 may comprise an amino acid sequence as set forth in SEQ ID NO. 29; the L-FR1 may comprises an amino acid sequence as set forth in SEQ ID NO.32, the L-FR2 may comprises an amino acid sequence as set forth in SEQ ID NO.26, the L-FR3 may comprises an amino acid sequence as set forth in SEQ ID NO.28 and the L-FR4 may comprises an amino acid sequence as set forth in SEQ ID NO.30; the VL may comprise an amino acid sequence as set forth in SEQ ID NO.10. And the antibody comprises the above VH and VL may be TN36-8.

For example, the VH may comprise HCDR1-3 and H-FR1-4, the HCDR1 may comprise an amino acid sequence as set forth in SEQ ID NO. 13, the HCDR2 may comprise an amino acid sequence as set forth in SEQ ID NO. 15, and the HCDR3 may comprise an amino acid sequence as set forth in SEQ ID NO. 17; the H-FR1 may comprises an amino acid sequence as set forth in SEQ ID NO.12, the H-FR2 may comprises an amino acid sequence as set forth in SEQ ID NO.14, the H-FR3 may comprises an amino acid sequence as set forth in SEQ ID NO.19, and the H-FR4 may comprises an amino acid sequence as set forth in SEQ ID NO.18; the VH may comprise an amino sequence as set forth in SEQ ID NO.4; the heavy chain constant region may comprise an amino acid sequence as set forth in SEQ ID NO. 48. And the VL may comprise LCDR1-3 and L-FR1-4, the LCDR1 may comprise an amino acid sequence as set forth in SEQ ID NO. 25, the LCDR2 may comprise an amino acid sequence as set forth in SEQ ID NO. 27, and the LCDR3 may comprise an sequence as set forth in SEQ ID NO.33, the L-FR2 may comprises an amino acid sequence as set forth in SEQ ID NO.26, the L-FR3 may comprises an amino acid sequence as set forth in SEQ ID NO.34 and the L-FR4 may comprises an amino acid sequence as set forth in SEQ ID NO.30; the VL may comprise an amino acid sequence as set forth in SEQ ID NO.11. And the antibody comprises the above VH and VL may be TN36-9.

For example, the VH may comprise HCDR1-3 and H-FR1-4, the HCDR1 may comprise an amino acid sequence as set forth in SEQ ID NO. 13, the HCDR2 may comprise an amino acid sequence as set forth in SEQ ID NO. 15, and the HCDR3 may comprise an amino acid sequence as set forth in SEQ ID NO. 17; the H-FR1 may comprises an amino acid sequence as set forth in SEQ ID NO.12, the H-FR2 may comprises an amino acid sequence as set forth in SEQ ID NO.14, the H-FR3 may comprises an amino acid sequence as set forth in SEQ ID NO.20, and the H-FR4 may comprises an amino acid sequence as set forth in SEQ ID NO.21; the VH may comprise an amino sequence as set forth in SEQ ID NO.4; the heavy chain constant region may comprise an amino acid sequence as set forth in SEQ ID NO. 48. And the VL may comprise LCDR1-3 and L-FR1-4, the LCDR1 may comprise an amino acid sequence as set forth in SEQ ID NO. 25, the LCDR2 may comprise an amino acid sequence as set forth in SEQ ID NO. 27, and the LCDR3 may comprise an amino acid sequence as set forth in SEQ ID NO. 29; the L-FR1 may comprises an amino acid sequence as set forth in SEQ ID NO.24, the L-FR2 may comprises an amino acid sequence as set forth in SEQ ID NO.26, the L-FR3 may comprises an amino acid sequence as set forth in SEQ ID NO.28 and the L-FR4 may comprises an amino acid sequence as set forth in SEQ ID NO.30; the VL may comprise an amino acid sequence as set forth in SEQ ID NO.8. And the antibody comprises the above VH and VL may be TN36-10.

For example, the VH may comprise HCDR1-3 and H-FR1-4, the HCDR1 may comprise an amino acid sequence as set forth in SEQ ID NO. 13, the HCDR2 may comprise an amino acid sequence as set forth in SEQ ID NO. 15, and the HCDR3 may comprise an amino acid sequence as set forth in SEQ ID NO. 17; the H-FR1 may comprises an amino acid sequence as set forth in SEQ ID NO.12, the H-FR2 may comprises an amino acid sequence as set forth in SEQ ID NO.14, the H-FR3 may comprises an amino acid sequence as set forth in SEQ ID NO.20, and the H-FR4 may comprises an amino acid sequence as set forth in SEQ ID NO.21; the VH may comprise an amino sequence as set forth in SEQ ID NO.4; the heavy chain constant region may comprise an amino acid sequence as set forth in SEQ ID NO. 48. And the VL may comprise LCDR1-3 and L-FR1-4, the LCDR1 may comprise an amino acid sequence as set forth in SEQ ID NO. 25, the LCDR2 may comprise an amino acid sequence as set forth in SEQ ID NO. 27, and the LCDR3 may comprise an amino acid sequence as set forth in SEQ ID NO. 29; the L-FR1 may comprises an amino acid sequence as set forth in SEQ ID NO.24, the L-FR2 may comprises an amino acid sequence as set forth in SEQ ID NO.31, the L-FR3 may comprises an amino acid sequence as set forth in SEQ ID NO.28 and the L-FR4 may comprises an amino acid sequence as set forth in SEQ ID NO.30; the VL may comprise an amino acid sequence as set forth in SEQ ID NO.9. And the antibody comprises the above VH and VL may be TN36-11.

For example, the VH may comprise HCDR1-3 and H-FR1-4, the HCDR1 may comprise an amino acid sequence as set forth in SEQ ID NO. 13, the HCDR2 may comprise an amino acid sequence as set forth in SEQ ID NO. 15, and the HCDR3 may comprise an amino acid sequence as set forth in SEQ ID NO. 17; the H-FR1 may comprises an amino acid sequence as set forth in SEQ ID NO.12, the H-FR2 may comprises an amino acid sequence as set forth in SEQ ID NO.14, the H-FR3 may comprises an amino acid sequence as set forth in SEQ ID NO.20, and the H-FR4 may comprises an amino acid sequence as set forth in SEQ ID NO.21; the VH may comprise an amino sequence as set forth in SEQ ID NO.4; the heavy chain constant region may comprise an amino acid sequence as set forth in SEQ ID NO. 48. And the VL may comprise LCDR1-3 and L-FR1-4, the LCDR1 may comprise an amino acid sequence as set forth in SEQ ID NO. 25, the LCDR2 may comprise an amino acid sequence as set forth in SEQ ID NO. 27, and the LCDR3 may comprise an amino acid sequence as set forth in SEQ ID NO. 29; the L-FR1 may comprises an amino acid sequence as set forth in SEQ ID NO.32, the L-FR2 may comprises an amino acid sequence as set forth in SEQ ID NO.26, the L-FR3 may comprises an amino acid sequence as set forth in SEQ ID NO.28 and the L-FR4 may comprises an amino acid sequence as set forth in SEQ ID NO.30; the VL may comprise an amino acid sequence as set forth in SEQ ID NO.10. And the antibody comprises the above VH and VL may be TN36-12.

For example, the VH may comprise HCDR1-3 and H-FR1-4, the HCDR1 may comprise an amino acid sequence as set forth in SEQ ID NO. 13, the HCDR2 may comprise an amino acid sequence as set forth in SEQ ID NO. 15, and the HCDR3 may comprise an amino acid sequence as set forth in SEQ ID NO. 17; the H-FR1 may comprises an amino acid sequence as set forth in SEQ ID NO.12, the H-FR2 may comprises an amino acid sequence as set forth in SEQ ID NO.14, the H-FR3 may comprises an amino acid sequence as set forth in SEQ ID NO.20, and the H-FR4 may comprises an amino acid sequence as set forth in SEQ ID NO.21; the VH may comprise an amino sequence as set forth in SEQ ID NO.4; the heavy chain constant region may comprise an amino acid sequence as set forth in SEQ ID NO. 48. And the VL may comprise LCDR1-3 and L-FR1-4, the LCDR1 may comprise an amino acid sequence as set forth in SEQ ID NO. 25, the LCDR2 may comprise an amino acid sequence as set forth in SEQ ID NO. 27, and the LCDR3 may comprise an amino acid sequence as set forth in SEQ ID NO. 29; the L-FR1 may comprises an amino acid sequence as set forth in SEQ ID NO.33, the L-FR2 may comprises an amino acid sequence as set forth in SEQ ID NO.26, the L-FR3 may comprises an amino acid sequence as set forth in SEQ ID NO.34 and the L-FR4 may comprises an amino acid sequence as set forth in SEQ ID NO.30; the VL may comprise an amino acid sequence as set forth in SEQ ID NO.11. And the antibody comprises the above VH and VL may be TN36-13.

For example, the VH may comprise HCDR1-3 and H-FR1-4, the HCDR1 may comprise an amino acid sequence as set forth in SEQ ID NO. 13, the HCDR2 may comprise an amino acid sequence as set forth in SEQ ID NO. 15, and the HCDR3 may comprise an amino acid sequence as set forth in SEQ ID NO. 17; the H-FR1 may comprises an amino acid sequence as set forth in SEQ ID NO.22, the H-FR2 may comprises an amino acid sequence as set forth in SEQ ID NO.14, the H-FR3 may comprises an amino acid sequence as set forth in SEQ ID NO.16, and the H-FR4 may comprises an amino acid sequence as set forth in SEQ ID NO.23; the VH may comprise an amino sequence as set forth in SEQ ID NO.4; the heavy chain constant region may comprise an amino acid sequence as set forth in SEQ ID NO. 48. And the VL may comprise LCDR1-3 and L-FR1-4, the LCDR1 may comprise an amino acid sequence as set forth in SEQ ID NO. 25, the LCDR2 may comprise an amino acid sequence as set forth in SEQ ID NO. 27, and the LCDR3 may comprise an amino acid sequence as set forth in SEQ ID NO. 29; the L-FR1 may comprises an amino acid sequence as set forth in SEQ ID NO.24, the L-FR2 may comprises an amino acid sequence as set forth in SEQ ID NO.26, the L-FR3 may comprises an amino acid sequence as set forth in SEQ ID NO.28 and the L-FR4 may comprises an amino acid sequence as set forth in SEQ ID NO.30; the VL may comprise an amino acid sequence as set forth in SEQ ID NO.8. And the antibody comprises the above VH and VL may be TN36-14.

For example, the VH may comprise HCDR1-3 and H-FR1-4, the HCDR1 may comprise an amino acid sequence as set forth in SEQ ID NO. 13, the HCDR2 may comprise an amino acid sequence as set forth in SEQ ID NO. 15, and the HCDR3 may comprise an amino acid sequence as set forth in SEQ ID NO. 17; the H-FR1 may comprises an amino acid sequence as set forth in SEQ ID NO.22, the H-FR2 may comprises an amino acid sequence as set forth in SEQ ID NO.14, the H-FR3 may comprises an amino acid sequence as set forth in SEQ ID NO.16, and the H-FR4 may comprises an amino acid sequence as set forth in SEQ ID NO.23; the VH may comprise an amino sequence as set forth in SEQ ID NO.4; the heavy chain constant region may comprise an amino acid sequence as set forth in SEQ ID NO. 48. And the VL may comprise LCDR1-3 and L-FR1-4, the LCDR1 may comprise an amino acid sequence as set forth in SEQ ID NO. 25, the LCDR2 may comprise an amino acid sequence as set forth in SEQ ID NO. 27, and the LCDR3 may comprise an amino acid sequence as set forth in SEQ ID NO. 29; the L-FR1 may comprises an amino acid sequence as set forth in SEQ ID NO.24, the L-FR2 may comprises an amino acid sequence as set forth in SEQ ID NO.31, the L-FR3 may comprises an amino acid sequence as set forth in SEQ ID NO.28 and the L-FR4 may comprises an amino acid sequence as set forth in SEQ ID NO.30; the VL may comprise an amino acid sequence as set forth in SEQ ID NO.9. And the antibody comprises the above VH and VL may be TN36-15.

For example, the VH may comprise HCDR1-3 and H-FR1-4, the HCDR1 may comprise an amino acid sequence as set forth in SEQ ID NO. 13, the HCDR2 may comprise an amino acid sequence as set forth in SEQ ID NO. 15, and the HCDR3 may comprise an amino acid sequence as set forth in SEQ ID NO. 17; the H-FR1 may comprises an amino acid sequence as set forth in SEQ ID NO.22, the H-FR2 may comprises an amino acid sequence as set forth in SEQ ID NO.14, the H-FR3 may comprises an amino acid sequence as set forth in SEQ ID NO.16, and the H-FR4 may comprises an amino acid sequence as set forth in SEQ ID NO.23; the VH may comprise an amino sequence as set forth in SEQ ID NO.4; the heavy chain constant region may comprise an amino acid sequence as set forth in SEQ ID NO. 48. And the VL may comprise LCDR1-3 and L-FR1-4, the LCDR1 may comprise an amino acid sequence as set forth in SEQ ID NO. 25, the LCDR2 may comprise an amino acid sequence as set forth in SEQ ID NO. 27, and the LCDR3 may comprise an sequence as set forth in SEQ ID NO.32, the L-FR2 may comprises an amino acid sequence as set forth in SEQ ID NO.26, the L-FR3 may comprises an amino acid sequence as set forth in SEQ ID NO.28 and the L-FR4 may comprises an amino acid sequence as set forth in SEQ ID NO.30; the VL may comprise an amino acid sequence as set forth in SEQ ID NO.10. And the antibody comprises the above VH and VL may be TN36-16.

For example, the VH may comprise HCDR1-3 and H-FR1-4, the HCDR1 may comprise an amino acid sequence as set forth in SEQ ID NO. 13, the HCDR2 may comprise an amino acid sequence as set forth in SEQ ID NO. 15, and the HCDR3 may comprise an amino acid sequence as set forth in SEQ ID NO. 17; the H-FR1 may comprises an amino acid sequence as set forth in SEQ ID NO.22, the H-FR2 may comprises an amino acid sequence as set forth in SEQ ID NO.14, the H-FR3 may comprises an amino acid sequence as set forth in SEQ ID NO.16, and the H-FR4 may comprises an amino acid sequence as set forth in SEQ ID NO.23; the VH may comprise an amino sequence as set forth in SEQ ID NO.4; the heavy chain constant region may comprise an amino acid sequence as set forth in SEQ ID NO. 48. And the VL may comprise LCDR1-3 and L-FR1-4, the LCDR1 may comprise an amino acid sequence as set forth in SEQ ID NO. 25, the LCDR2 may comprise an amino acid sequence as set forth in SEQ ID NO. 27, and the LCDR3 may comprise an amino acid sequence as set forth in SEQ ID NO. 29; the L-FR1 may comprises an amino acid sequence as set forth in SEQ ID NO.33, the L-FR2 may comprises an amino acid sequence as set forth in SEQ ID NO.26, the L-FR3 may comprises an amino acid sequence as set forth in SEQ ID NO.34 and the L-FR4 may comprises an amino acid sequence as set forth in SEQ ID NO.30; the VL may comprise an amino acid sequence as set forth in SEQ ID NO.11. And the antibody comprises the above VH and VL may be TN36-17.

For example, the VH may comprise HCDR1-3 and H-FR1-4, the HCDR1 may comprise an amino acid sequence as set forth in SEQ ID NO. 13, the HCDR2 may comprise an amino acid sequence as set forth in SEQ ID NO. 15, and the HCDR3 may comprise an amino acid sequence as set forth in SEQ ID NO. 17; the H-FR1 may comprises an amino acid sequence as set forth in SEQ ID NO.22, the H-FR2 may comprises an amino acid sequence as set forth in SEQ ID NO.14, the H-FR3 may comprises an amino acid sequence as set forth in SEQ ID NO.16, and the H-FR4 may comprises an amino acid sequence as set forth in SEQ ID NO.21; the VH may comprise an amino sequence as set forth in SEQ ID NO.6; the heavy chain constant region may comprise an amino acid sequence as set forth in SEQ ID NO. 48. And the VL may comprise LCDR1-3 and L-FR1-4, the LCDR1 may comprise an amino acid sequence as set forth in SEQ ID NO. 25, the LCDR2 may comprise an amino acid sequence as set forth in SEQ ID NO. 27, and the LCDR3 may comprise an amino acid sequence as set forth in SEQ ID NO. 29; the L-FR1 may comprises an amino acid sequence as set forth in SEQ ID NO.24, the L-FR2 may comprises an amino acid sequence as set forth in SEQ ID NO.26, the L-FR3 may comprises an amino acid sequence as set forth in SEQ ID NO.28 and the L-FR4 may comprises an amino acid sequence as set forth in SEQ ID NO.30; the VL may comprise an amino acid sequence as set forth in SEQ ID NO.8. And the antibody comprises the above VH and VL may be TN36-18.

For example, the VH may comprise HCDR1-3 and H-FR1-4, the HCDR1 may comprise an amino acid sequence as set forth in SEQ ID NO. 13, the HCDR2 may comprise an amino acid sequence as set forth in SEQ ID NO. 15, and the HCDR3 may comprise an amino acid sequence as set forth in SEQ ID NO. 17; the H-FR1 may comprises an amino acid sequence as set forth in SEQ ID NO.22, the H-FR2 may comprises an amino acid sequence as set forth in SEQ ID NO.14, the H-FR3 may comprises an amino acid sequence as set forth in SEQ ID NO.16, and the H-FR4 may comprises an amino acid sequence as set forth in SEQ ID NO.21; the VH may comprise an amino sequence as set forth in SEQ ID NO.6; the heavy chain constant region may comprise an amino acid sequence as set forth in SEQ ID NO. 48. And the VL may comprise LCDR1-3 and L-FR1-4, the LCDR1 may comprise an amino acid sequence as set forth in SEQ ID NO. 25, the LCDR2 may comprise an amino acid sequence as set forth in SEQ ID NO. 27, and the LCDR3 may comprise an amino acid sequence as set forth in SEQ ID NO. 29; the L-FR1 may comprises an amino acid sequence as set forth in SEQ ID NO.24, the L-FR2 may comprises an amino acid sequence as set forth in SEQ ID NO.31, the L-FR3 may comprises an amino acid sequence as set forth in SEQ ID NO.28 and the L-FR4 may comprises an amino acid sequence as set forth in SEQ ID NO.30; the VL may comprise an amino acid sequence as set forth in SEQ ID NO.9. And the antibody comprises the above VH and VL may be TN36-19.

For example, the VH may comprise HCDR1-3 and H-FR1-4, the HCDR1 may comprise an amino acid sequence as set forth in SEQ ID NO. 13, the HCDR2 may comprise an amino acid sequence as set forth in SEQ ID NO. 15, and the HCDR3 may comprise an amino acid sequence as set forth in SEQ ID NO. 17; the H-FR1 may comprises an amino acid sequence as set forth in SEQ ID NO.22, the H-FR2 may comprises an amino acid sequence as set forth in SEQ ID NO.14, the H-FR3 may comprises an amino acid sequence as set forth in SEQ ID NO.16, and the H-FR4 may comprises an amino acid sequence as set forth in SEQ ID NO.21; the VH may comprise an amino sequence as set forth in SEQ ID NO.6; the heavy chain constant region may comprise an amino acid sequence as set forth in SEQ ID NO. 48. And the VL may comprise LCDR1-3 and L-FR1-4, the LCDR1 may comprise an amino acid sequence as set forth in SEQ ID NO. 25, the LCDR2 may comprise an amino acid sequence as set forth in SEQ ID NO. 27, and the LCDR3 may comprise an amino acid sequence as set forth in SEQ ID NO. 29; the L-FR1 may comprises an amino acid sequence as set forth in SEQ ID NO.32, the L-FR2 may comprises an amino acid sequence as set forth in SEQ ID NO.26, the L-FR3 may comprises an amino acid sequence as set forth in SEQ ID NO.28 and the L-FR4 may comprises an amino acid sequence as set forth in SEQ ID NO.30; the VL may comprise an amino acid sequence as set forth in SEQ ID NO.10. And the antibody comprises the above VH and VL may be TN36-20.

For example, the VH may comprise HCDR1-3 and H-FR1-4, the HCDR1 may comprise an amino acid sequence as set forth in SEQ ID NO. 13, the HCDR2 may comprise an amino acid sequence as set forth in SEQ ID NO. 15, and the HCDR3 may comprise an amino acid sequence as set forth in SEQ ID NO. 17; the H-FR1 may comprises an amino acid sequence as set forth in SEQ ID NO.22, the H-FR2 may comprises an amino acid sequence as set forth in SEQ ID NO.14, the H-FR3 may comprises an amino acid sequence as set forth in SEQ ID NO.16, and the H-FR4 may comprises an amino acid sequence as set forth in SEQ ID NO.21; the VH may comprise an amino sequence as set forth in SEQ ID NO.6; the heavy chain constant region may comprise an amino acid sequence as set forth in SEQ ID NO. 48. And the VL may comprise LCDR1-3 and L-FR1-4, the LCDR1 may comprise an amino acid sequence as set forth in SEQ ID NO. 25, the LCDR2 may comprise an amino acid sequence as set forth in SEQ ID NO. 27, and the LCDR3 may comprise an amino acid sequence as set forth in SEQ ID NO. 29; the L-FR1 may comprises an amino acid sequence as set forth in SEQ ID NO.33, the L-FR2 may comprises an amino acid sequence as set forth in SEQ ID NO.26, the L-FR3 may comprises an amino acid sequence as set forth in SEQ ID NO.34 and the L-FR4 may comprises an amino acid sequence as set forth in SEQ ID NO.30; the VL may comprise an amino acid sequence as set forth in SEQ ID NO.11. And the antibody comprises the above VH and VL may be TN36-21.

In the present disclosure, said antibody is a rabbit antibody, a chimeric antibody or humanized antibody.

In the present disclosure, said antibody or the antigen binding fragment thereof further comprises a heavy chain constant region of a human IgG and/or a light chain constant region of a human antibody.

In the present disclosure, said antibody or the antigen binding fragment thereof comprises a human IgG1, IgG2, IgG3, or IgG4 heavy chain constant region.

In the present disclosure, said antibody or the antigen binding fragment thereof comprises a human Kappa or Lambda light chain constant region.

In the present disclosure, said antibody or the antigen binding fragment thereof is IgG, IgM, IgA, IgD or IgE.

In the present disclosure, said antibody or the antigen binding fragment thereof may comprise a heavy chain constant region and/or a light chain constant region. The heavy chain constant region may comprise an amino acid sequence as set forth in SEQ ID NO: 48, and the light chain constant region may comprise an amino acid sequence as set forth in SEQ ID NO: 49.

In the present disclosure, said antibody or the antigen binding fragment thereof is: a full-length antibody, or a scFv, Fv, sdFv, Fab, Fab′ or F(ab′)2.

In the present disclosure, said TNFR2 is a human TNFR2 and/or a Cynomolgus TNFR2.

In some cases, said antibody or the antigen binding fragment thereof may comprise a polypeptide having a sequence identity of at least 80% with any one of the above amino acid sequences in the present disclosure. For example, said polypeptide may have a sequence identity of 80% (e.g., at least 85%, at least 90%, at least 91%, at least 92%, at least 93%, at least 94%, at least 95%, at least 96%, at least 97%, at least 98%, at least 99% or higher) to the antibody or antigen binding fragment thereof.

The term “percent (%) sequence identity,” as used in the context of polypeptide sequences identified herein, generally refers to the percentage of amino acid residues or nucleotides in a query sequence that are identical with the amino acid residues or nucleotides of a second, reference polypeptide sequence or a portion thereof, after aligning the sequences and introducing gaps, if necessary, to achieve the maximum percent sequence identity, and not considering any conservative substitutions as part of the sequence identity. Alignment for purposes of determining percent amino acid/nucleotide sequence identity can be achieved in various ways that are within the skill in the art, for instance, using publicly available computer software such as BLAST, BLAST-2, ALIGN, NEEDLE or Megalign (DNASTAR) software. Those skilled in the art can determine appropriate parameters for measuring alignment, including any algorithms needed to achieve maximal alignment over the full length of the sequences being compared. Percent identity may be measured over the length of an entire defined polypeptide/polynucleotide sequence, or may be measured over a shorter length, for example, over the length of a fragment taken from a larger, defined polypeptide/polynucleotide sequence. It is understood that any fragment length supported by the sequences shown herein, in the tables, figures or Sequence Listing, may be used to describe a length over which percentage identity may be measured.

In another aspect, the present disclosure provided an isolated nucleic acid molecule, which encodes a heavy chain of the antibody or the antigen binding fragment thereof.

The isolated nucleic acids may comprise one or more nucleic acid molecules, with each encoding the antigen binding protein. For example, the isolated nucleic acids may comprise at least two nucleic acid molecules, with one encoding the antibody heavy chain or a fragment thereof, and one encoding the antibody light chain or a fragment thereof.

The isolated nucleic acid or isolated nucleic acids may be synthesized using recombinant techniques well known in the art. For example, the isolated nucleic acid or isolated nucleic acids may be synthesized with an automated DNA synthesizer. Standard recombinant DNA and molecular cloning techniques include those described by Sambrook, J., Fritsch, E. F. and Maniatis, T. Molecular Cloning: A Laboratory Manual; Cold Spring Harbor Laboratory Press: Cold Spring Harbor, (1989) (Maniatis) and by T. J. Silhavy, M. L. Bennan, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, pub. by Greene Publishing Assoc. and Wiley-Interscience (1987). Briefly, the subject nucleic acids may be prepared from genomic DNA fragments, cDNAs, and RNAs, all of which may be extracted directly from a cell or recombinantly produced by various amplification processes including but not limited to PCR and RT-PCR.

Direct chemical synthesis of nucleic acids typically involves sequential addition of 3′-blocked and 5′-blocked nucleotide monomers to the terminal 5′-hydroxyl group of a growing nucleotide polymer chain, wherein each addition is effected by nucleophilic attack of the terminal 5′-hydroxyl group of the growing chain on the 3′-position of the added monomer, which is typically a phosphorus derivative, such as a phosphotriester, phosphoramidite, or the like. See for example, Matteuci et al., Tet. Lett. 521:719 (1980); U.S. Pat. No. 4,500,707 to Caruthers et al.; and U.S. Pat. Nos. 5,436,327 and 5,700,637 to Southern et al.

In another aspect, the present disclosure provided an isolated nucleic acid, which encodes a light chain of the antibody or the antigen binding fragment thereof.

In another aspect, the present disclosure provided an isolated nucleic acid, which encodes the antibody or the antigen binding fragment thereof.

In another aspect, the present disclosure provided an expression vector, which contains said nucleic acid.

The expression vector may be any linear nucleic acids, plasmids, phagemids, cosmids, RNA vectors, viral vectors and the like. Non-limiting examples of a viral vector may include a retrovirus, an adenovirus and an adeno-associated virus. In some embodiments, the expression vector may be a plasmid.

An expression vector may be suitable for use in particular types of host cells and not suitable for use in others. For example, the expression vector may be introduced into the host organism, which may be then monitored for viability and expression of any genes/polynucleotides contained in the expression vector.

The expression vector may also comprise one or more selectable marker genes that, upon expression, confer one or more phenotypic traits useful for selecting or otherwise identifying host cells that carry the expression vector. Non-limiting examples of suitable selectable markers for eukaryotic cells include dihydrofolate reductase and neomycin resistance.

The subject vectors can be introduced into a host cell stably or transiently by a variety of established techniques. Lipid complexes, liposomes, and dendrimers may also be employed to transfect the host cells.

In another aspect, the present disclosure provided a host cell, which contains said isolated nucleic acid and/or the expression vector.

The host cell may express the antigen binding protein of the present disclosure. The cell may be a eukaryotic cell or a prokaryotic cell. An appropriate cell may be transformed or transfected with the nucleic acid(s) or vector(s) of the present disclosure and utilized for the expression and/or secretion of the antigen binding protein.

In the present disclosure, said host cell produces said antibody or the antigen binding fragment thereof.

In another aspect, the present disclosure provided a composition, which comprises said antibody or the antigen binding fragment thereof, and optionally a pharmaceutically acceptable carrier.

As used herein, “pharmaceutically acceptable carrier” may comprise any and all preservatives, wetting agents, emulsifying agents and dispersing agents. Prevention of presence of microorganisms may be ensured both by sterilization procedures, supra, and by the inclusion of various antibacterial and antifungal agents. It may also be desirable to include isotonic agents, sodium chloride, and the like into the compositions.

Pharmaceutical compositions typically must be sterile and stable under the conditions of manufacture and storage. The composition may be formulated as a solution, microemulsion, liposome, or other ordered structure suitable to high drug concentration. The carrier may be a solvent or dispersion medium containing, and suitable mixtures thereof. The proper fluidity can be maintained, for example, by the use of a coating such as lecithin, by the maintenance of the required particle size in the case of dispersion and by the use of surfactants.

In another aspect, the present disclosure provided a method for preparing a TNFR2 antibody or the antigen binding fragment thereof, which comprises: culturing said host cell and recovering said antibody or said antigen binding fragment thereof.

The method optionally may further comprise harvesting the antigen binding protein of the present disclosure.

In another aspect, the present disclosure provided a method for treating a tumor and/or for inhibiting the growth of tumor cells expressing TNFR2, which comprises: administering said antibody or an antigen binding fragment thereof, said nucleic acid, and/or said expression vector, said host cell, or said composition.

In another aspect, the present disclosure provided said antibody or an antigen binding fragment thereof, said nucleic acid, and/or said expression vector, said host cell, or said composition, for use in treating a tumor and/or for inhibiting the growth of tumor cells expressing TNFR2.

In another aspect, the present disclosure provided a use of said antibody or an antigen binding fragment thereof, said nucleic acid, and/or said expression vector, said host cell, or said composition in treating a tumor and/or for inhibiting the growth of tumor cells expressing TNFR2.

In another aspect, the present disclosure provided a use of said antibody or an antigen binding fragment thereof, said nucleic acid, and/or said expression vector, said host cell, or said composition for preparing a medicament, said medicament is used for treating a tumor and/or inhibiting the growth of tumor cells expressing TNFR2.

In another aspect, the present disclosure provided a use of said antibody or an antigen binding fragment thereof, said nucleic acid, and/or said expression vector, said host cell, or said composition in preparing a drug for treating a tumor and/or for inhibiting the growth of tumor cells expressing TNFR2.

### EXAMPLES

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); m., intramuscular(ly); i.p., intraperitoneal(ly); s. c., subcutaneous(ly); and the like.

### Example 1: Rabbit Immunization

Standard 77-day rabbit immunization protocol was used for anti-TNFR2 rabbit polyclonal antibody generation. In brief, two NZW SPF rabbits (New Zealand white rabbits that are specific pathogen-free) were immunized with 0.5 mg recombinant TNFR2-His protein (Acro Biosystem, TN2-H5227) in Complete Freund's Adjuvant (CFA) on day 1, boosted with 0.25 mg of TNFR2-His antigen in Incomplete Freund's Adjuvant (IFA) on day 21,35, 49 and 63. Pre-immune bleed (5 mL per rabbit) were collected on day 0, Immune bleed (˜25 mL per rabbit) were collected on day 70 to determine antibody titer, on day 77 spleens were harvested for phage library construction.

### Example 2: TNFR2 Focused scFv Phage Library Construction

Total RNA from spleens of the immunized animal was isolated using RNeasy Plus Mini Kit (Qiagen) according to manufacturer's instructions. cDNA was synthesized from lug total RNA using Invitrogen SuperScript IV First-Strand Synthesis kit according to the manufacturer's instruction (ThermoFisher). Rabbit VH and VL regions were PCR-amplified using forward and reverse primers.

The VH and VL PCR products were gel-purified and used as a templet for 2nd round of overlapping PCR using confidential forward primer with Sad restriction site and reverse primer with SpeI restriction site, The final PCR product were digested with the restriction enzyme Sad and SpeI (NEB), followed by ligation with the Oak engineered pIII phage library plasmid that has been pre-digested with the same enzyme. Ten micrograms of the ligation products were used to transform 0.6 mL of E. coli TG1 competent cells (Invitrogen) by electroporation according to the manufacturer's instruction. The transformed TG1 cells were recovered for 1 h at 37° C., shaking at 250 rpm, then plate on ×YT agar plates overnight. The clones were scrapped onto 250 ml 2YT medium with 2% glucose and inoculated into 1 L of 2×YT media and cultured for additional 1 h at 37° C., shaking at 250 rpm. Afterwards, 1×10¹⁰ helper phage M13KO7 (NEB) was added to the TG1 cell culture, followed by incubation at 37° C. overnight. The cells were centrifuged at 8500 g for 15 min, and the supernatant containing the phage particles was collected and mixed with ¼ volume of sterile polyethylene glycol (PEG) 8000/NaCl solution (20% PEG in 2.5 M NaCl solution). The phage/PEG solution mixture was incubated on ice for 1 h and centrifuged at 10000 g for 15 min. The final phage pellet was resuspended in 20 mL of PBS buffer containing 20% glycerol, aliquoted in 1 mL volume size and stored at −80° C.

### Example 3: Phage Panning

This example shows a method for in solution panning of phage library. Biotinylated TNFR2-His protein was prepared using Thermo Scientific EZ-Link NHS-PEG4 Biotinylation Kit according to manufacture protocol, then mixed with streptavidin beads at molar ratio of 4:1 at 4° C. for 1 hour, TNFR2 coated streptavidin beads were washed 3 times with PBS containing 2% BSA and used to pan TNFR2 immunized scFv library. In brief, TNFR2 coated streptavidin beads and phage solution was incubated at 37° C. for 1 h. After extensive washing with PBS buffer, TNFR2 specific phage binders were eluted from the streptavidin beads by pH 2.0 citric acid buffer and were immediately neutralized with Tris-HCl buffer pH 8.0. The eluted output phage was re-amplified by re-infection of fresh TG1 cells, and the re-amplified phage was used as the input for the next round of panning. After three to four rounds of panning, single colonies were randomly picked from the output phage infected TG1 cells, monoclonal phage ELISA was performed to identify TNFR2 specific binders.

### Example 4: Monoclonal Phage ELISA

This example shows a method for determination of target binding capability of phage by ELISA. A 96-well ELISA plate was coated with hTNFR2-His and blocked with PBS buffer containing 2% BSA at 4.0 overnight, 1004, of pre-blocked phage solution was added to the plate and incubated at 37° C. for 30 min. After the plate was washed twice with PBS buffer containing 0.05% Tween 20, phage binding was detected by HRP-conjugated anti-M13 antibody (GE Healthcare). Top binders were picked and the corresponding phagemids in TG1 cell were isolated and subjected to DNA sequencing to obtain VH/VL sequence. The sequences of TN1-36_VH and TN1-36_VL are set forth as SEQ ID NO: 1 and SEQ ID NO: 2, respectively. Antibody numbering and CDR annotation are performed according to IMGT scheme respectively.

### Example 5: Generation of Chimeric Anti-TNFR2 Antibody

This example shows a method for generating a chimeric anti-TNFR2 antibody. The variable heavy chains of TN1-36_VH was genetically fused to a human IgG1 constant region to produce chimeric TN1-36 hIgG1. Similarly, the variable light chains of TN1-36_VL was genetically fused to a human IgG1 kappa constant domain. These DNA constructs were cloned into the pcDNA3.1(+) expression vector. The heavy chain vector constructs together with the respective light chain vector constructs were transiently co-expressed in the CHO cell line to generate chimeric antibodies. The protein from the CHO supernatant was purified using Capturem™ Protein A Miniprep Columns (Takarabio, #635717) following manufacturer's instructions. The purified antibody was subjected to a series of in vitro characterizations described in other examples.

### Example 6: Humanization of Chimeric Anti-TNFR2 Antibody

This example shows a method for humanization of antibody. The chimeric anti-TNFR2 antibody prepared in example 5 (TN36 for short) was humanized by inserting the rabbit CDRs into human germline frameworks. The destination frameworks were engineered according to the modelling top 5 germline frameworks according to the reducing potential immunogenicity.

First, TN36 heavy and light variable regions were aligned with human variable sequences from IMGT to identify human germline genes for CDR grafting. Top 5 frameworks were chosen for grafting the variable light chain and heavy chain based on highest framework homology. Second, a 3-D structural model was generated to evaluate any differences between the human and rabbit framework residues. Framework residues will be back mutated to corresponding rabbit framework residues if they are predicted to influence CDR binding. Framework back mutations were evaluated in the variable light chains and variable heavy chains, respectively. As a result, humanized VH constructs AL2, AL3, AL4, AL5, AL6 were generated and humanized VL constructs AL8, AL9, AL10, AL11 were generated.

The sequences of AL2 is set forth as SEQ ID NO: 3; the sequences of AL3 is set forth as SEQ ID NO: 4; the sequences of AL4 is set forth as SEQ ID NO: 5; the sequences of AL5 is set forth as SEQ ID NO: 6; the sequences of AL6 is set forth as SEQ ID NO: 7; and the sequences of AL8 is set forth as SEQ ID NO: 8; the sequences of AL9 is set forth as SEQ ID NO: 9; the sequences of AL10 is set forth as SEQ ID NO: 10; the sequences of AL11 is set forth as SEQ ID NO: 11.

These humanized VH and VL constructs were cloned into human IgG1 Fc and human Ck pcDNA3.1(+) based expression vectors respectively, then combined in a pairwise fashion as shown in Table 1 and transiently co-expressed in CHO cell line to generate humanized antibodies. The supernatant from each expression was collected on day 5 and analyzed for TNFR2 binding.

TABLE 1
Anti-TNFR2 antibody candidates and the
light chains and heavy chains thereof

| C | L | | | |
|---|---|---|---|---|
| HC | AL8 | AL9 | AL10 | AL11 |
| AL2 | TN36-2 | TN36-3 | TN36-4 | TN36-5 |
| AL3 | TN36-6 | TN36-7 | TN36-8 | TN36-9 |
| AL4 | TN36-10 | TN36-11 | TN36-12 | TN36-13 |
| AL5 | TN36-14 | TN36-15 | TN36-16 | TN36-17 |
| AL6 | TN36-18 | TN36-19 | TN36-20 | TN36-21 |

During the work of humanization of TN36, TN36 was renamed as TN36-1.

### Example 7: Ranking Candidates by ELISA

This example shows a method for ranking binding affinity of purified anti-TNFR2 antibody candidates produced from example 6. Briefly, 50 μl of 5.0 μg/ml TNFR2-His (Acro Biosystem, TN2-H5227), diluted in PBS pH7.4 were used to coat 96 well ELISA plate at 4° C. overnight. After washing the wells 3 times with 0.05% Triton in 1×PBS (PBST), the wells were blocked with 300 μl 1×PBS with 3% non-fat milk per well (blocking buffer) at 4° C. overnight. the wells were washed 3 times with 1×PBST.

Anti-TNFR2 antibody antibodies were added in 100 μl (in blocking buffer) with amount of 0.006 μg, 0.06 μg/well, and incubate at 4° C. overnight. Wash the wells 4 times with 1×PBST. 100 μl HRP-conjugated Goat anti-Human Kappa Light Chain Secondary Antibody (Invitrogen Cat #A18853) diluted 1:2,000 in blocking buffer were added to each well, and incubate at RT for 1 hour. Wash the wells 4 times with 1×PBST. Then 100 μl Thermo Scientific 1-Step Ultra TMB-ELISA substrate solution (cat #34028) was then added into each well, and was incubated at RT for 5 min. Avoid light during the reaction. Finally 100 μl of 2 M H₂SO₄ were added to each well to stop reaction and Read OD at 450 nm.

The results can be seen in FIG. 1, and the results showed that 20 anti-TNFR2 antibody candidates produced from example 6 were confirmed to bind it target TNFR2. These antibodies displayed various binding towards its target TNFR2 with TN36-17 as the strongest one and TN36-12 the weakest.

### Example 8: Characterization of Target TNFR2 Binding

ELISA assay was used to evaluate the binding affinity of purified anti-TNFR2 antibody candidates produced from example 6 to tumor necrosis factor receptors (TNFRs) from multiple species.

Human TNFR2 (Acro Biosystem, TN2-H5227), Human TNFR1 (Acro Biosystem, TN1-H5222), Cynomolgus TNFR2 (Sino Biological, 90102-CO8H) or mouse TNFR2 (R&D systems, 426-R2-050/CF) was coated on high binding polystyrene flat bottom micro-titer plates (Thermo Scientific, 3455) in PBS (PH=7.4, Gibco,10010-031) at 1 μg/mL and 100 μL per well. Plates were incubated at 4° C. overnight.

Plates were warmed up to room temperature the next day and washed with 200 μL PBS containing 0.1% Tween 20 (Life technologies, 003005) per well for 3 times. Plates were blocked with 200 μL PBS buffer containing 1% BSA (Fisher Scientific, BP1600-100) per well for 1 hour at 37° C. Antibodies was titrated from 5 μg/mL in a 5-fold dilution series, distributed 100 μL per well and incubated at 37° C. for 1 hour. Human IgG1 isotype control (BioxCell, BE0297) was used as negative control. Plates were washed with 200 μL PBS containing 0.1% Tween 20 (Life technologies, 003005) per well for 3 times. Goat anti-Human IgG Fc Secondary Antibody-HRP conjugate (Invitrogen, A18817) were distributed at 0.5μg/mL and 100 μL per well to detect bound antibodies. After 1-hour incubation of secondary antibody at 37° C., plates were washed with 200 μL PBS containing 0.1% Tween 20 (Life technologies, 003005) per well for 3 times. Plates were developed using TMB Substrate Solution (eBioscience, 00-4201-56) and stopped with ELISA stop solution (Invitrogen, SSO4). The level of bound antibodies was determined by reading absorbance at 450 nm. Mouse anti-human TNFR1 antibody (ThermoFisher Scientific, MA1-81005) and goat anti mouse IgG secondary antibody-HRP conjugate (Invitrogen, 62-5520) were used as positive control for human TNFR1 detection. Armenian Hamster anti-mouse TNFR2 antibody (InVivoMab, BE0247) and HRP-Goat anti-Armenian Hamster IgG (H+L) Secondary Antibody (Invitrogen, PA1-32045) were used as positive control for mouse TNFR2 detection.

For the binding affinity to rat TNFR2 test, rat TNFR2 (R&D systems, 8348-R2-050) was coated on high binding polystyrene flat bottom micro-titer plates (Thermo Scientific, 3455) in PBS (PH=7.4, Gibco,10010-031) at 1 μg/ml and 100 μL per well. Plates were incubated at 4° C. overnight. Antibodies was titrated from 100 μg/ml in a 5-fold dilution series, and distributed 100 μL, per well and incubated at 37° C. for 1 hour. Human IgG1 isotype control (BioxCell, BE0297) was used as negative control. TNFR2 Polyclonal Antibody (Invitrogen, PAS-80159) and HRP conjugated Goat anti-Rabbit IgG Fc Secondary Antibody (Invitrogen, A16116) were used as positive control for rat TNFR2 detection.

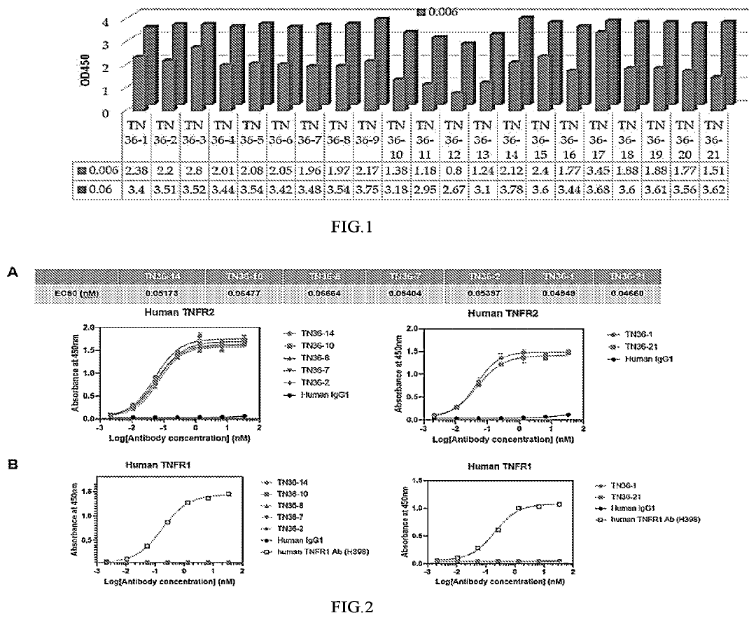

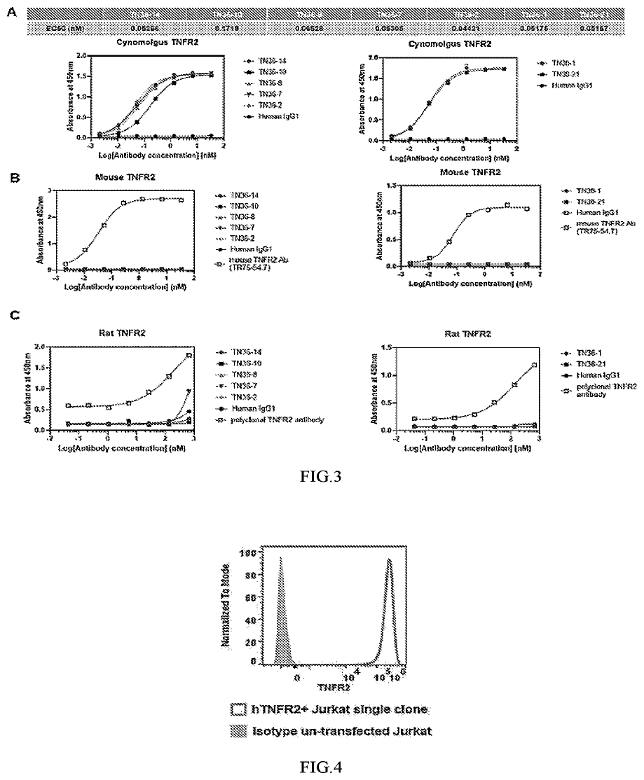

The results can be seen in FIG. 2A-2B, and the results showed that the anti-TNFR2 antibody candidates bind strongly and specifically to human (FIG. 2A) and cyno TNFR2 (FIG. 3A), but not to human TNFR1 (FIG. 2B), mouse or rat TNFR2 (FIG. 3B-3C).

### Example 9: TNFR2 Jurkat Reporter Stable Cell Line Establishment

This example shows a method for establishment of TNFR2 overexpressing stable cell line. Full length human TNFR2 was cloned into pLVX-EFla-IRES-Puro vector (Takarabio, 631253). Lentivirus production and target cell transfection were performed according to manufacturer's instructions. Briefly, human TNFR2-lentivector was mixed with ViraPower™ Packaging Mix (Invitrogen, K4975-00) and transfect 293T cell line (Sigma-Aldrich, 12022001) using Lipofectamine 2000 Reagent (Invitrogen, 11668-019) to produce a lentiviral stock. Jurkat E6-1 cells (ATCC, TIB-152) were transduced with human TNFR2-lentivirus in the presence of 8 μg/mL polybrene (EMD, Millipore, TR-1003-G) and selected with 1 μg/mL puromycin (Gibco, A11138-03).

The pool of surviving cells was expanded. For identification of cells expressing high levels of human TNFR2, the surviving cells were blocked with human TruStain FcX™ (Biolegend, 422302) for 10 minutes at room temperature according to manufacturer's instructions. Then cells were stained with phycoerythrin (PE) conjugate anti-human TNFR2 antibody (ThermoFisher Scientific, TNFR7504) for 30 minutes at 4° C. Single clones were generated from the highest-expressing TNFR2 cells by single cell plate sorting on cell sorter (Sony, SH800). Clone NO.3 expressing high level of human TNFR2 was selected (FIG. 4) and used for subsequent cell surface binding and in vitro functional assays.

### Example 10: Cell Surface Binding

FACS analysis was used to evaluate the binding of purified anti-TNFR2 antibody candidates produced from example 6 to cell surface TNFR2 target. Anti-human TNFR2 antibodies and negative control IgG were biotinylated using a EZ-Link™ Micro Sulfo-NHS-Biotinylation Kit (Thermo Fisher Scientific, 21925) according to manufacturer's instructions. Biotinylated antibodies (100 μg/mL) were then incubated with Halt™ Protease Inhibitor Cocktail (100X, Thermo Scientific, 1862209) and 80 μg/mL Streptavidin-R-Phycoerythrin (PE) conjugate (Invitrogen, S21388). Antibodies were stored at 4° C. until use. 5×10⁵ Clone No.3 of human TNFR2⁺ Jurkat cells were distributed each well and blocked with human TruStain FcX™ (Biolegend, 422302) for 10 minutes at room temperature before stained with a serially diluted PE conjugated anti-human TNFR2 antibodies or hIgG1 starting from 50 μg/mL in a 5-fold dilution fashion for 30 minutes at 4° C. PE-anti hTNFR2 (Clone MR2-1, Thermo fisher #TNFR7504) was used as positive control. The geometric mean fluorescent intensity was measured on Sony SA3800 cell analyzer. Data were analyzed using Flowjo Software (Tree star Inc).

The results can be seen in FIG. 5A-5B, and the results showed that the anti-TNFR2 antibody candidates bind to target TNFR2 expressed on cell surface.

### Example 11: Ligand Binding Competition Assay

The potential to compete with ligand TNFα for receptor binding of purified anti-TNFR2 antibody candidates produced from example 6 can be evaluated by competition ELISA experiment.

Human TNFα (Gibco, PHC3011) was biotinylated using an EZ-Link™ Micro Sulfo-NHS-Biotinylation Kit (ThermoFisher Scientific, 21925) according to manufacturer's instructions. Human TNFR2 (Acro Biosystem, TN2-H5227) was coated on high binding polystyrene flat bottom micro-titer plates (Thermo Scientific, 3455) in PBS (PH=7.4, Gibco, 10010-031) at 1 μg/mL and 100 μL per well. Plates were incubated at 4° C. overnight. Plates were warmed up to room temperature the next day and washed with 200 μL PBS containing 0.1% Tween 20 (Life technologies, 003005) per well for 3 times. Plates were then blocked with 200 μL PBS buffer containing 1% BSA (Fisher Scientific, BP1600-100) per well for 1 hour at 37° C. The anti-TNFR2 antibody candidates were titrated from 10 μg/mL in a 5-fold dilution series and distributed 50 μL per well. Human IgG1 isotype control (BioxCell, BE0297) was used as negative control. 1 hour after the incubation of the anti-TNFR2 antibody candidates, 50 μL biotinylated human TNFα was added per well to achieve the final TNFα concentration at 100 ng/ml. After another 1-hour incubation, plates were washed with 200 μL PBS containing 0.1% Tween 20 (Life technologies, 003005) per well for 3 times. Streptavidin-HRP conjugate (Thermo Scientific, N504) were distributed at 0.5 μg/mL and 100 μL per well to detect bound biotinylated human TNFα. After 1-hour incubation of secondary antibody at 37° C., plates were washed with 200 μL PBS containing 0.1% Tween 20 (Life technologies, 003005) per well for 3 times. Plates were developed using TMB Substrate Solution (eBioscience,00-4201-56) and stopped with ELISA stop solution (Invitrogen, SSO4). The level of bound biotinylated human TNFα was determined by reading absorbance at 450 nm.

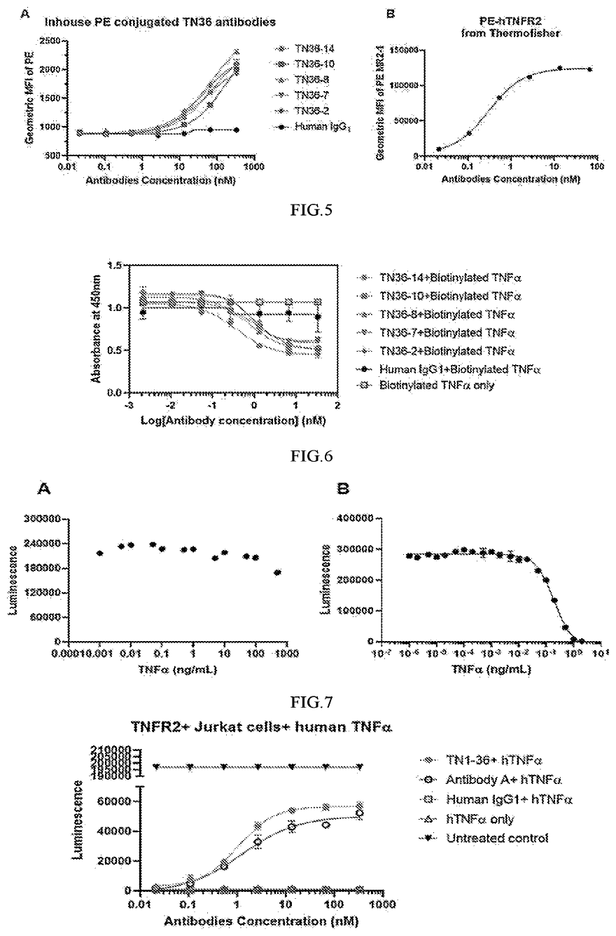

The results can be seen in FIG. 6, and the results showed that the anti-TNFR2 antibody candidates can partially compete with ligand TNFα for TNFR2 binding, and this ligand blocking activity of the anti-TNFR2 antibody candidates may account for their cellular function described in later section.

One of anti-TNFR2 antibody candidates produced from example 6 has been selected for further characterization and development and designated as Antibody A in the following sessions.

### Example 12: In Vitro Function Assay

The cellular function of antibodies of the present disclosure can be evaluated by measuring TNFα induced hTNFR2 Jurkat cell death.

Parental Jurkat E6-1 cells or TNFR2⁺ Jurkat cell line Clone NO.3 cells were plated in 96 well plates with 10⁴ cells in 100 μL complete RPMI medium per well. Gradient concentration of human TNFα (Gibco, PHC3011) was added. The cell viability was measured after 24 hours with Promega CellTiter-Glo® Luminescent Cell Viability Assay (Promega, G7570) according to manufacturer's instructions. Under this experiment condition, parental Jurkat E6-1 cells are resistant to TNFα induced cell death up to 1000 ng/ml TNFα (FIG. 7A), while TNFR2⁺ Jurkat cells are highly sensitive to TNFα induced cell death with 99% cell killing is achieved at TNFα concentration as low as 2 ng/ml (FIG. 7B). This TNFα induced cell death is apparently mediated by TNFR2 overexpressed on Jurkat cell as parental Jurkat is not sensitive. Presumably TNFR2 blocking antibody or antagonist antibody will alleviate TNFα induced cell death and TNFR2 agonist antibody will enhance TNFα induced cell death. This assay provides a good opportunity to evaluate in vitro cellular function of antibodies of present application.

Anti-human TNFR2 antibodies were added to the Clone NO.3 cells by dose titration along with constant human TNFα (0.5 ng/ml). The cell viability was measured after 24 hours with Promega CellTiter-Glo® Luminescent Cell Viability Assay (Promega, G7570). human IgG1 were used as a negative control for this assay.

In experiments performed essentially as described above, the Antibody A can rescue TNFR2⁺ Jurkat cells from TNFα induced cell death (FIG. 8). And the result indicated that the Antibody A may antagonize TNFα through blocking TNFα/TNFR2 signaling and subsequentially alleviate TNFα induced cell death.

### Example 13: Anti-TNFR2 Antibody A Doesn't Agonize TNFR2 Receptor

Antibody against TNF family members is prone to be agonistic given the fact that crosslinking of TNF family members with bivalent antibody might trigger their signaling without need of ligand TNFα. The ability of anti-TNFR2 antibodies of present disclosure to agonize TNFR2 receptor can be evaluated using TNFR2 Jurkat cell-based assay.

A 1:5 serially diluted anti-human TNFR2 antibodies or human IgG1 starting from 50 μg/mL were incubated with constant 10 μg/mL AffiniPure Goat Anti-Human IgG (Jackson Immuno Research Laboratories, Inc, Cata #109-005-098) at 37° C. for 2 hours. The mixture was then added to Clone NO.3 TNFR2⁺ Jurkat cells (104 cells per well) in total volume of 100 μL. The cell viability was measured after 24 hours with Promega CellTiter-Glo® Luminescent Cell Viability Assay (Promega, G7570).

The results can be seen in FIG. 9, and the results showed that the Antibody A itself has no effect on Jurkat cell death therefore cannot agonize TNFR2 receptor even in the presence of antibody crosslinking.

### Example 14: Colo205 Assay

TNFR2 was reported to be elevated on tumor cells from different type of cancer patients. Anti-TNFR2 antibody may also have impact on tumor cell proliferation/death directly. The impact on tumor cell death of antibodies of the present disclosure can be evaluated using Colo205 cell line (ATCC, CCL-222).

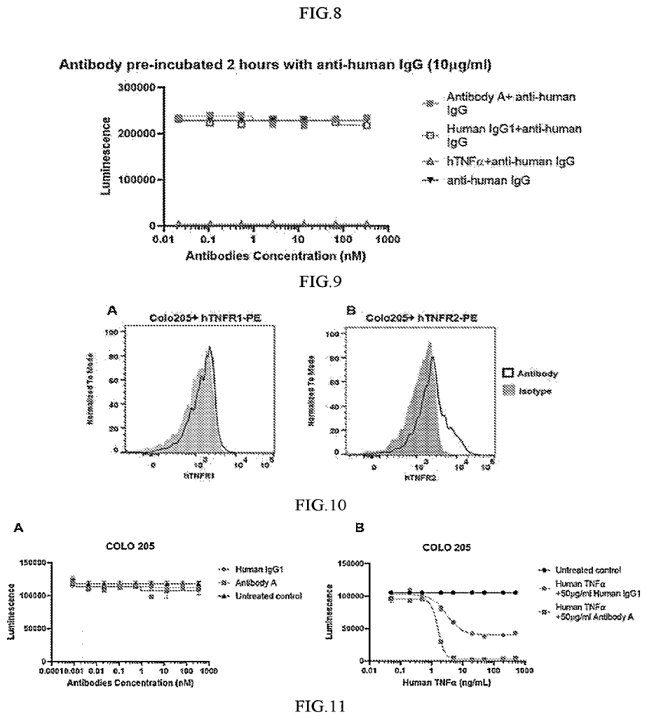

To determine whether TNFR1 or TNFR2 is expressed on Colo205 cell line. Overnight cultured Colo205 cells were blocked with human TruStain FcX™ (Biolegend, 422302) for 10 minutes at room temperature according to manufacturer's instructions. Cells were then stained with PE anti-human CD120a Antibody (Biolegend ,369904) or PE anti-human TNFR2 antibody (ThermoFisher Scientific, TNFR7504) for 30 minutes at 4° C. PE Mouse IgG2a (Invitrogen, PAS-33240) or PE Mouse IgG1 kappa (Invitrogen,12-4714-82) were used as isotype control. Data was obtained on Sony SA3800 cell analyzer and analyzed using Flowjo Software (Tree star Inc). FACS data suggests Colo205 expresses TNFR2 modestly (FIG. 10A) while no TNFR1 expression (FIG. 10B).

To test whether the Antibody A can lead to Colo205 cell death directly. Colo205 cells were plated at 5,000 cell per well in complete RPMI medium in 96 well plates. a 1:5 serially diluted the Antibody A or human IgG1 starting from 50 μg/mL were added the next day. The cell viability was measured with Promega CellTiter-Glo® Luminescent Cell Viability Assay (Promega, G7570) after 72 hours after addition of antibodies. At all concentrations tested, the Antibody A itself has no effect on cell viability (FIG. 11A).

To determine whether the Antibody A enhances TNF-mediated cell killing activity towards Colo205 cell line, 50 μg/mL the Antibody A and IgG1 were added to the Colo205 cells along with a serially diluted human TNFα next day. The cell viability was measured after 72 hours after human TNFα addition with Promega CellTiter-Glo® Luminescent Cell Viability Assay (Promega, G7570).

In experiments performed essentially as described above, TNFα can induce Colo205 cell death in a dose-dependent manner, while the Antibody A enhances TNFα-dependent cell killing activity towards Colo205 cell line (FIG. 11B).

### Example 15: PK (Pharmacokinetic) Study of the Antibody A in Mice

The single dose PK of the Antibody A in plasma can be evaluated by administering the antibody at doses of 1, 3, 10 mg/kg to C57BL/6 WT female mice (n=3) via the tail vein. Injection volume is 10 μl/g. Blood samples were collected from the cervical vein without anesthesia and mixed with heparin sodium at 3 days before injection and 1 hr, 24 hrs, 48 hrs, 96 hrs, 144 hrs, 192 hrs and 384 hrs after injection. Plasma samples was obtained by centrifuging the blood (10,000 rpm, 4° C., min) and stored at −80° C. until use.

The Antibody A concentrations in plasma samples were measured using a standard sandwich ELISA. In brief, 100 ng His tagged human TNFR2 ECD (Acro, #TN2-H5227) were coated on ELISA plate overnight at 4° C. The plates were washed 4 times with wash buffer (PBS+0.05% (v/v) TWEEN20) and blocked with PBS containing 1% BSA at room temperature for 1 hr followed by wash 4 times with wash buffer. Plasma samples were added to plate and incubated at 37 for 1 hr. After wash 4 times with wash buffer, 100 μl 1:5000 diluted Peroxidase-conjugated AffiniPure F(ab′)2 Fragment Goat Anti-Human IgG Fc (Jackson Immuno Research, #109-036-098) in PBS containing 1% BSA were added to each well and incubated at 37 for 1 hr. After wash 4 times, 100 μl substrate TMB were added and incubated at room temperature for 15 minutes in the dark, 100 μl stop solution were added to stop the reaction and read OD450 on plate reader.

In vivo PK data were analyzed using WinNonlin (Phoenix™, version 8.0, Pharsight). A Noncompartment model (moment) analysis was performed to obtain the following parameters: T1/2 (half-life), Area under the plasma concentration-time curve AUC0-384, AUC0-inf, Cl (clearance), and Vss (Volume of distribution Steady-State), MRT (Mean residence time of the unchanged drug in the systemic circulation).

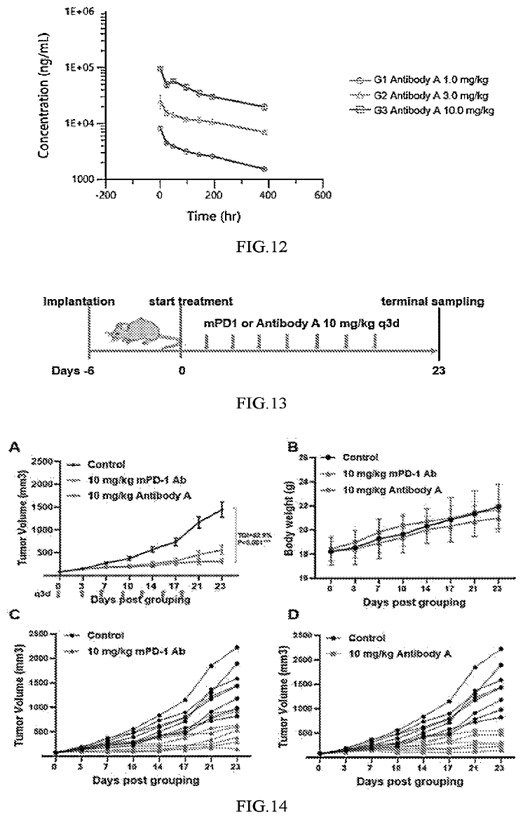

In experiments performed essentially as described above, the Antibody A showed a linear clearance following IV dosing in mice. Mean T1/2 ranges from 256.49 hrs at 10 mg/kg and 339.00 hrs at 3 mg/kg. Mean Cl ranges from 0.4 ml/hr/kg to 0.59 ml/hr/kg. Within dose range of 1.0 mg/kg to 10.0 mg/kg), AUC0-384 h displayed a linear correlation with dose level (FIG. 12 and Table 2). This linear clearance PK property and slow clearance rate made it interesting to further evaluate the Antibody A PK profile in cynomolgus monkey, a more relevant specie model.

TABLE 2

| | T½ | AUC0-384 h | AUC0-∞ | Cl | Vss_obs | MRTlast |
|---|---|---|---|---|---|---|
| Group: TA/Dose(mg/kg) | (hr) | (hr*ng/mL) | (hr*ng/mL) | (mL/hr/kg) | (mL/kg) | (hr) |
| G1: Antibody A/1.0 | 271.79 | 1089898.55 | 1690398.46 | 0.59 | 219.47 | 146.43 |
| G2: Antibody A/3.0 | 339.00 | 4250156.32 | 7649693.88 | 0.40 | 191.63 | 158.39 |
| G3: Antibody A/10.0 | 256.49 | 13654175.92 | 21113664.55 | 0.48 | 172.46 | 146.21 |

### Example 16: In Vivo Efficacy of the Antibody A

The antibody of the present disclosure can be evaluated for in vivo anti-tumor activity using B-hTNFR2 MC38 mouse model. In this TNFR2 humanized mice, the extracellular domain of mouse TNFRSF1B gene was replaced by human TNFRSF1B counterparts. Basal leukocyte subpopulations of TNFR2 humanized mice were comparable to those of wild-type mice, including T/NK cells, B cells, DC, granulocytes, and monocytes/macrophages. Human TNFR2 was detectable on the Tregs in spleen as well. TNFR2 humanized mice is a useful tool for in vivo efficacy evaluation of anti-hTNFR2 antibodies (biocytogen 2020 AACR poster #5050).

Murine colon cancer MC38 cells (5×10⁵) were subcutaneously implanted into homozygous B-hTNFR2 mice (female, 9-10-week-old, n=8). Mice were grouped when tumor volume reached approximately 80-100 mm³, at which time they were treated with an i. p. injection of PBS, 10 mg/kg anti-mPD1 antibody, 10 mg/kg the Antibody A every 3 days for 7 doses in total as shown in FIG. 13. Animal well-being and behavior, including grooming and ambulation, are monitored at least twice per week. Body weight and tumor volume are measured twice a week. The antitumor efficacy is expressed as TGITV ratio in percent and calculated as summarized below:

TGITV (%)=[1−(Ti−T0)/(Ci−C0)]×100%

Where Ti=mean tumor volume of drug-treated group on the final day of the study, T0=mean tumor volume of drug-treated group on initiating day of dosing, Ci=mean tumor volume of control group on the final day of the study, C0=mean tumor volume of control group on initiating day of dosing. The statistical significance of experimental data was evaluated using the Student's t-test between treatment groups to isotype control.

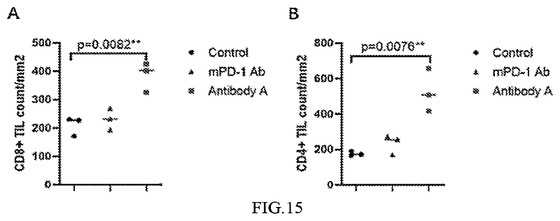

In experiments performed essentially as described above, treatment with the Antibody A significantly inhibits tumor growth in the humanized mice, comparable to treatment with anti-mPD1 antibody in the same study (FIG. 14A). treatment with the Antibody A results in a TGITV=82.9% with p<0.001 (Table 3). In the meantime, treatment with the Antibody A did not cause body weight loss at all comparing to control group (FIG. 14B). Individual tumor measurements plot for each group were shown in FIG. 14C-14D. At the end of study, plasma samples from all 3 treatment groups were collected for subsequent cytokine/chemokine analysis, tumor samples were divided by half for RNAseq and IHC analysis respectively.

TABLE 3

| | Tumor Volume (mm³) ^{a} | | | |
|---|---|---|---|---|
| | Before | After dosing | TGI_{TV} | |
| Test articles | dosing | Day23 | (%) | P ^{b} |
| PBS | 77 ± 2 | 1443 ± 164 | — | — |
| anti-mouse PD-1 | 77 ± 2 | 561 ± 100 | 64.5 | ***p < 0.001 |
| (CD279) | | | | |
| Antibody A | 77 ± 3 | 311 ± 54 | 82.9 | p < 0.001 |

^{a} mean ± SEM;
^{b} T-test of tumor volume between drug-treated group and control group on day 23.
*P < 0.05,
**P < 0.01,
***P < 0.001

### Example 17: Tumor Infiltrating Lymphocytes after the Antibody A Treatment

Tumor infiltrating lymphocytes (TILs) are usually associated with good prognosis in different tumor types. Tumors with high TILs are considered as inflamed tumor or hot tumor and usually respond well to immune checkpoint inhibitor or other types of immunotherapy. The effect of Anti-hTNFR2 Antibody A on TILs count can be evaluated by CD4 and CD8 Immunohistochemistry (IHC).

Immunohistochemistry was performed using primary antibodies against CD4⁺ (Cell signaling #25229S, Rabbit IgG1), CD8⁺ (Cell signaling #98941, Rabbit IgG1). Formalin-fixed, paraffin-embedded tissues sections (3-4 μm thick) were taken on 3-aminopropyltriethoxysilane (APTS) coated glass slides then de-paraffinised in xylene followed by hydration in graded ethanol. Specimens were heated at 100° C. for 20 min in 0.01M citrate buffer (pH 6.0) using an EZ antigen retriever system (Biogenex, USA) to retrieve antigen. Endogenous peroxidase was blocked by treatment with 0.3% hydrogen peroxide for 5 min, non-specific binding sites were blocked with a protein block for 5 min. Sections were covered with primary antibody followed by incubation in moist chamber overnight at 4° C. After treatment with primary antibody, slides were washed with TBS (pH 7.4), blocked with post primary block at room temperature for 30 min. Sections were then washed twice in TBS followed by incubation with Novolink polymer for 30 min at room temperature. After washing with TBS three times, sections were treated with DAB chromogen (3, 3′-diaminobenzidine tetrahydrochloride) in the dark for 5-10 min. Sections were counterstained with hematoxylin, dehydrated with ethanol and xylene, and mounted permanently with Di-n-butylPhthalate in Xylene (DPX). Negative control slides omitting the primary antibody were included in all batches.

Scoring of immune stained positive TILs was done independently by at least two pathologists. CD4⁺, and CD8⁺ TILs were counted in five randomly selected fields at 40× magnification and the counts were averaged. The cell counts for each treatment were summarized in Table 4-5 (each treatment had been repeated for third times) and density (No./mm²) are plotted using Graphpad software (FIG. 15 A-B, representing the tumor infiltrating CD8⁺ T cell and CD4⁺ T cell, respectively).

These results showed that the treatment with the Antibody A significantly increases tumor infiltrating CD4/8⁺ T cell, while anti-PD1 antibody had much less effect of tumor infiltrating CD4/8+ T cell in the same study. This observation might imply the underlying anti-tumor mechanism of TNFR2 antibody and PD-1 antibody is different, which might suggest a possibility of combination of these two agents for a significant good efficacy.

TABLE 4
CD8 IHC

| | Positive | Measured | Measured | |
|---|---|---|---|---|
| | count | area | area | Density |
| Treatment | No. | (μm2) | (mm2) | (No./mm2) |
| Negative control | 32929 | 144766672 | 144.766672 | 227.4625751 |
| Negative control | 27849 | 162383408 | 162.383408 | 171.501512 |
| Negative control | 30329 | 131616208 | 131.616208 | 230.4351452 |
| Anti-mPD1 10 | 25378 | 94484392 | 94.484392 | 268.5946267 |
| mg/kg | | | | |
| Anti-mPD1 10 | 24295 | 104765392 | 104.765392 | 231.8990989 |
| mg/kg | | | | |
| Anti-mPD1 10 | 8392 | 43563800 | 43.5638 | 192.637006 |
| mg/kg | | | | |
| Antibody A 10 | 13976 | 32834864 | 32.834864 | 425.6451313 |
| mg/kg | | | | |
| Antibody A 10 | 10041 | 24930020 | 24.93002 | 402.7674266 |
| mg/kg | | | | |
| Antibody A 10 | 13689 | 42049612 | 42.049612 | 325.5440264 |
| mg/kg | | | | |

TABLE 5
CD4 IHC

| | Positive | Measured | Measured | |
|---|---|---|---|---|
| | count | area | area | Density |
| Treatment | No. | (μm2) | (mm2) | (No./mm2) |
| Negative control | 27109 | 1.6E+08 | 163.117 | 166.193 |
| Negative control | 27906 | 1.6E+08 | 161.872 | 172.396 |
| Negative control | 24831 | 1.3E+08 | 130.017 | 190.982 |
| Anti-mPD1 10 | 16404 | 9.5E+07 | 95.4544 | 171.852 |
| mg/kg | | | | |
| Anti-mPD1 10 | 28611 | 1.1E+08 | 105.191 | 271.992 |
| mg/kg | | | | |
| Anti-mPD1 10 | 11498 | 4.5E+07 | 45.0115 | 255.446 |
| mg/kg | | | | |
| Antibody A 10 | 14344 | 3.4E+07 | 34.4196 | 416.739 |
| mg/kg | | | | |
| Antibody A 10 | 17339 | 2.6E+07 | 26.3773 | 657.346 |
| mg/kg | | | | |
| Antibody A 10 | 21869 | 4.3E+07 | 42.9998 | 508.583 |
| mg/kg | | | | |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. An antibody or an antigen binding fragment thereof capable of specifically binding to Tumor necrosis factor receptor type 2 (TNFR2), which comprises a heavy chain variable region (VH), a light chain variable region (VL) or both,
wherein said VH comprises at least one heavy chain complementarity determining region (HCDR), which comprises an amino acid sequence selected from the group consisting of the amino acid sequences as set forth in SEQ ID NO: 13, SEQ ID NO: 15 and SEQ ID NO: 17, and
wherein the VL comprises at least one light chain complementarity determining region (LCDR), which comprises an amino acid sequence selected from the group consisting of the amino acid sequences as set forth in SEQ ID NO: 25, SEQ ID NO: 27 and SEQ ID NO: 29.

2. The antibody or the antigen binding fragment thereof according to claim 1, wherein said VH comprises an HCDR3, which comprises the amino acid sequence of SEQ ID NO: 17; or
said VH comprises an HCDR2, which comprises the amino acid sequence of SEQ ID NO: 15; or
said VH comprises HCDR1, which comprises the amino acid sequence of SEQ ID NO: 13; or
said VH comprises HCDR1 and HCDR3, which comprise the amino acid sequences of SEQ ID NO: 13 and SEQ ID NO: 17 respectively; or
said VH comprises HCDR2 and HCDR3, which comprise the amino acid sequences of SEQ ID NO: 15 and SEQ ID NO: 17 respectively; or
said VH comprises HCDR1 and HCDR2, which comprise the amino acid sequences of SEQ ID NO: 13 and SEQ ID NO: 15 respectively; or
said VH comprises HCDR1, HCDR2 and HCDR3, which comprise the amino acid sequences of SEQ ID NO: 13, SEQ ID NO: 15 and SEQ ID NO: 17 respectively.

3-8. (canceled)

9. The antibody or the antigen binding fragment thereof according to claim 1, wherein said VH comprises HFR1, said HFR1 comprises the amino acid sequence of SEQ ID NO: 35; or
said VH comprises HFR1, which comprises the amino acid sequence as set forth in SEQ ID NO: 42 (Formula (I)), wherein X1 is selected from hydrophobic amino acids and/or aliphatic amino acids; or
said VH comprises HFR1, which is selected from the group consisting of the amino acid sequences as set forth in SEQ ID NO: 35, SEQ ID NO: 22 and SEQ ID NO: 12; or
said VH comprises HFR2, which comprises the amino acid sequence as set forth in SEQ ID NO: 36 or SEQ ID NO: 14; or
said VH comprises HFR3, which comprises the amino acid sequence of SEQ ID NO: 37; or
said VH comprises HFR3, which comprises the amino acid sequence as set forth in SEQ ID NO: 43 (Formula (II)), wherein the X2 is selected from hydrophilic amino acids, wherein the X3 is selected from hydrophobic amino acids and/or aliphatic amino acids, and wherein the X4 is selected from hydrophobic amino acids and/or aromatic amino acids; or
said VH comprises HFR3, which is selected from the group consisting of the amino acid sequences as set forth in SEQ ID NO: 37, SEQ ID NO: 20, SEQ ID NO: 19 and SEQ ID NO: 16; or
said VH comprises HFR4, which comprises the amino acid sequences as set forth in SEQ ID NO: 44 (Formula (III)), wherein the X5 is selected from any amino acids; or
said VH comprises HFR4, which is selected from the group consisting of the amino acid sequences as set forth in SEQ ID NO: 21, SEQ ID NO: 18 or SEQ ID NO: 23; or
said VH comprises HFR1 comprising the amino acid sequence of SEQ ID NO: 12, HFR2 comprising the amino acid sequence of SEQ ID NO: 14, HFR3 comprising the amino acid sequence of SEQ ID NO: 16, and HFR4 comprising the amino acid sequence of SEQ ID NO: 18; or
said VH comprises HFR1 comprising the amino acid sequence of SEQ ID NO: 12, HFR2 comprising the amino acid sequence of SEQ ID NO: 14, HFR3 comprising the amino acid sequence of SEQ ID NO: 19, and HFR4 comprising the amino acid sequence of SEQ ID NO: 18; or
said VH comprises HFR1 comprising the amino acid sequence of SEQ ID NO: 12, HFR2 comprising the amino acid sequence of SEQ ID NO: 14, HFR3 comprising the amino acid sequence of SEQ ID NO: 20, and HFR4 comprising the amino acid sequence of SEQ ID NO: 21; or
said VH comprises HFR1 comprising the amino acid sequence of SEQ ID NO: 22, HFR2 comprising the amino acid sequence of SEQ ID NO: 14, HFR3 comprising the amino acid sequence of SEQ ID NO: 16, and HFR4 comprising the amino acid sequence of SEQ ID NO: 23; or
said VH comprises HFR1 comprising the amino acid sequence of SEQ ID NO: 22, HFR2 comprising the amino acid sequence of SEQ ID NO: 14, HFR3 comprising the amino acid sequence of SEQ ID NO: 16, and HFR4 comprising the amino acid sequence of SEQ ID NO: 21.

10-22. (canceled)

23. The antibody or the antigen binding fragment thereof according to claim 1, wherein said VH comprises an amino acid sequence selected from the group consisting of the amino acid sequences as set forth in SEQ ID NO: 1, SEQ ID NO: 3, SEQ ID NO: 4, SEQ ID NO: 5, SEQ ID NO: 6 and SEQ ID NO: 7.

24. The antibody or the antigen binding fragment thereof according to claim 1, wherein said VL comprises LFR1, which comprise the amino acid sequences of SEQ ID NO: 38; or
said VL comprises LFR1, which comprises the amino acid sequences as set forth in SEQ ID NO: 45 (Formula (IV)), wherein the X6 is selected from hydrophobic amino acids and/or aliphatic amino acids, and wherein the X7 is selected from any amino acids; or
said VL comprises LFR2, which comprise the amino acid sequences of SEQ ID NO: 39; or
said VL comprises LFR2, which comprises the amino acid sequences as set forth in SEQ ID NO: 46 (Formula (V)), wherein the X8 is selected from small amino acid; or
said VL comprises LFR3, which comprises the amino acid sequences of SEQ ID NO: 40; or
said VL comprises LFR3, which comprises the amino acid sequences as set forth in SEQ ID NO: 47 (Formula (VI)), wherein the X9 is selected from negatively-charged amino acids or hydrophilic amino acids; or
said VL comprises LFR4, which comprises the amino acid sequences of SEQ ID NO: 41 or SEQ ID NO: 30.

25-30. (canceled)

31. The antibody or the antigen binding fragment thereof according to claim 1, wherein said VL comprises LCDR3, which comprises the amino acid sequence of SEQ ID NO: 29; or
said VL comprises LCDR2, which comprises the amino acid sequence of SEQ ID NO: 27;
said VL comprises LCDR1, which comprises the amino acid sequence of SEQ ID NO: 25; or
said VL comprises LCDR1 and LCDR3, which comprise the amino acid sequences of SEQ ID NO: 25 and SEQ ID NO: 29 respectively; or
said VL comprises LCDR2 and LCDR3, which comprise the amino acid sequences of SEQ ID NO: 27 and SEQ ID NO: 29 respectively; or
said VL comprises LCDR1 and LCDR2, which comprise the amino acid sequences of SEQ ID NO: 25 and SEQ ID NO: 27 respectively; or
said VL comprises LCDR1, LCDR2 and LCDR3, which comprise the amino acid sequences of SEQ ID NO: 25, SEQ ID NO: 27 and SEQ ID NO: 29 respectively.

32-37. (canceled)

38. The antibody or the antigen binding fragment thereof according to claim 31,
wherein said VL comprises LFR1 selected from the group consisting of the amino acid sequences as set forth in SEQ ID NO: 24,
LFR2 selected from the group consisting of the amino acid sequences as set forth in SEQ ID NO: 26,
LFR3 selected from the group consisting of the amino acid sequences as set forth in SEQ ID NO: 28, and
LFR4 selected from the group consisting of the amino acid sequences as set forth in SEQ ID NO: 30; or
said VL comprises LFR1 which comprises the amino acid sequence of SEQ ID NO: 24, LFR2 which comprises the amino acid sequence of SEQ ID NO: 31, LFR3 which comprises the amino acid sequence of SEQ ID NO: 28, and LFR4, which comprises the amino acid sequence of SEQ ID NO: 30; or
said VL comprises LFR1 which comprises the amino acid sequence of SEQ ID NO: 32, LFR2 which comprises the amino acid sequence of SEQ ID NO: 26, LFR3 which comprises the amino acid sequence of SEQ ID NO: 28, and LFR4 which comprises the amino acid sequence of SEQ ID NO: 30; or
said VL comprises LFR1 which comprises the amino acid sequence of SEQ ID NO: 33, LFR2 which comprises the amino acid sequence of SEQ ID NO: 26, LFR3 which comprises the amino acid sequence of SEQ ID NO: 34, and LFR4 which comprises the amino acid sequence of SEQ ID NO: 30.

39-41. (canceled)

42. The antibody or the antigen binding fragment thereof according to claim 1, wherein said VL comprises an amino acid sequence selected from the group consisting of the amino acid sequences as set forth in SEQ ID NO: 8-11.

43. The antibody or the antigen binding fragment thereof according to claim 1, wherein said VH comprises HCDR3 comprising the amino acid sequences of SEQ ID NO: 17, and wherein said VL comprises LCDR3 comprising the amino acid sequences of SEQ ID NO: 29; or
said VH comprises HCDR1, HCDR2 and HCDR3, which comprise the amino acid sequences of SEQ ID NO: 13, SEQ ID NO: 15 and SEQ ID NO: 17 respectively, and wherein said VL comprises LCDR1, LCDR2 and LCDR3, which comprise the amino acid sequences of SEQ ID NO: 25, SEQ ID NO: 27 and SEQ ID NO: 29 respectively; or
said VH comprises an amino acid sequence selected from the group consisting of the amino acid sequences as set forth in SEQ ID NO: 1, SEQ ID NO: 3, SEQ ID NO: 4, SEQ ID NO: 5, SEQ ID NO: 6 and SEQ ID NO: 7, and wherein said VL comprises an amino acid sequence selected from the group consisting of the amino acid sequences as set forth in SEQ ID NO: 2, SEQ ID NO: 8, SEQ ID NO: 9, SEQ ID NO: 10 and SEQ ID NO: 11; or
said VH comprises an amino acid sequence of SEQ ID NO: 1, and wherein said VL comprises an amino acid sequence of SEQ ID NO: 2; or
said VH comprises an amino acid sequence of SEQ ID NO: 3, and wherein said VL comprises an amino acid sequence of SEQ ID NO: 8; or
said VH comprises an amino acid sequence of SEQ ID NO: 3, and wherein said VL comprises an amino acid sequence of SEQ ID NO: 9; or
said VH comprises an amino acid sequence of SEQ ID NO: 3, and wherein said VL comprises an amino acid sequence of SEQ ID NO: 10; or
said VH comprises an amino acid sequence of SEQ ID NO: 3, and wherein said VL comprises an amino acid sequence of SEQ ID NO: 11; or
said VH comprises an amino acid sequence of SEQ ID NO: 4, and wherein said VL comprises an amino acid sequence of SEQ ID NO: 8; or
said VH comprises an amino acid sequence of SEQ ID NO: 4, and wherein said VL comprises an amino acid sequence of SEQ ID NO: 9; or
said VH comprises an amino acid sequence of SEQ ID NO: 4, and wherein said VL comprises an amino acid sequence of SEQ ID NO: 10; or
said VH comprises an amino acid sequence of SEQ ID NO: 4, and wherein said VL comprises an amino acid sequence of SEQ ID NO: 11; or
said VH comprises an amino acid sequence of SEQ ID NO: 5, and wherein said VL comprises an amino acid sequence of SEQ ID NO: 8; or
said VH comprises an amino acid sequence of SEQ ID NO: 5, and wherein said VL comprises an amino acid sequence of SEQ ID NO: 9; or
said VH comprises an amino acid sequence of SEQ ID NO: 5, and wherein said VL comprises an amino acid sequence of SEQ ID NO: 10; or
said VH comprises an amino acid sequence of SEQ ID NO: 5, and wherein said VL comprises an amino acid sequence of SEQ ID NO: 11; or
said VH comprises an amino acid sequence of SEQ ID NO: 6, and wherein said VL comprises an amino acid sequence of SEQ ID NO: 8; or
said VH comprises an amino acid sequence of SEQ ID NO: 6, and wherein said VL comprises an amino acid sequence of SEQ ID NO: 9; or
said VH comprises an amino acid sequence of SEQ ID NO: 6, and wherein said VL comprises an amino acid sequence of SEQ ID NO: 10; or
said VH comprises an amino acid sequence of SEQ ID NO: 6, and wherein said VL comprises an amino acid sequence of SEQ ID NO: 11; or
said VH comprises an amino acid sequence of SEQ ID NO: 7, and wherein said VL comprises an amino acid sequence of SEQ ID NO: 8; or
said VH comprises an amino acid sequence of SEQ ID NO: 7, and wherein said VL comprises an amino acid sequence of SEQ ID NO: 9; or
said VH comprises an amino acid sequence of SEQ ID NO: 7, and wherein said VL comprises an amino acid sequence of SEQ ID NO: 10; or
said VH comprises an amino acid sequence of SEQ ID NO: 7, and wherein said VL comprises an amino acid sequence of SEQ ID NO: 11.

44-66. (canceled)

67. The antibody or the antigen binding fragment thereof according to claim 1, wherein the antibody or the antigen binding fragment thereof is a rabbit antibody, a chimeric antibody or humanized antibody; or
the antibody or the antigen binding fragment thereof is IgG, IgM, IgA, IgD or IgE; or
the antibody or the antigen binding fragment thereof is:
1) a full-length antibody, or
2) a scFv, Fv, sdFv, Fab, Fab′ or F(ab′)₂.

68. The antibody or the antigen binding fragment thereof according to claim 1, wherein the antibody or the antigen binding fragment thereof further comprises a heavy chain constant region of a human IgG and/or a light chain constant region of a human antibody; or
the antibody or the antigen binding fragment thereof comprises a human IgG1, IgG2, IgG3, or IgG4 heavy chain constant region; or
the antibody or the antigen binding fragment thereof comprises a human Kappa or Lambda light chain constant region.

69-72. (canceled)

73. The antibody or the antigen binding fragment thereof according to claim 1, wherein said TNFR2 is a human TNFR2 and/or a Cynomolgus TNFR2.

74. An isolated nucleic acid molecule, which encodes a heavy chain of the antibody or the antigen binding fragment thereof according to claim 1.

75. An isolated nucleic acid, which encodes a light chain of the antibody or the antigen binding fragment thereof according to claim 1.

76. An isolated nucleic acid, which encodes the antibody or the antigen binding fragment thereof according to claim 1.

77. An expression vector, which contains the nucleic acid according to claim 7.

78. A host cell, which contains the isolated nucleic acid according to claim 74.

79. (canceled)

80. A composition, which comprises the antibody or the antigen binding fragment thereof according to claim 1, and optionally a pharmaceutically acceptable carrier.

81. A method for preparing a TNFR2 antibody or the antigen binding fragment thereof, which comprises: culturing the host cell of claim 78 and recovering the said antibody or said antigen binding fragment thereof.

82. A method for treating a tumor and/or for inhibiting the growth of tumor cells expressing TNFR2, which comprises: administering the antibody or antigen binding fragment thereof according to claim 1.

83-85. (canceled)


SEQUENCE LISTING (49 sequences)

SEQ ID NO: 1 - protein, 124 aa, Artificial Sequence
  VH
QQSVKESEGGLFKPTDTLTLTCTVSGFSLSSSAVSWVRQAPGNGLEWIGAIGSGGTSYYSNWAKSRSTITRDTNLNTVTLKMTSLTAADTATYFCARDPNSSDYPYGAYGMGLWGPGTLVTVSS

SEQ ID NO: 2 - protein, 111 aa, Artificial Sequence
  VL
AELDLTQTPASVEVAAGGTVTINCQVSQSISSYLSWYQQKPGQPPKLLIYYASTLASGVPSRFSGSGSGTEFTLTISGVQCDDAATYYCQQGYITSNVGNAFGGGTEVVVK

SEQ ID NO: 3 - protein, 124 aa, Artificial Sequence
  VH
EVQLVESGGGLIQPGGSLRLSCAASGFSLSSSAVSWVRQAPGKGLEWIGAIGSGGTSYYSNWAKSRFTISRDNSKNTLYLQMNSLRAEDTAVYYCARDPNSSDYPYGAYGMGLWGRGTLVTVSS

SEQ ID NO: 4 - protein, 124 aa, Artificial Sequence
  VH
EVQLVESGGGLIQPGGSLRLSCAASGFSLSSSAVSWVRQAPGKGLEWIGAIGSGGTSYYSNWAKSRFTISRDNSKNTLYLQMNSLRAEDTAVYFCARDPNSSDYPYGAYGMGLWGRGTLVTVSS

SEQ ID NO: 5 - protein, 124 aa, Artificial Sequence
  VH
EVQLVESGGGLIQPGGSLRLSCAASGFSLSSSAVSWVRQAPGKGLEWIGAIGSGGTSYYSNWAKSRFTISRDTSKNTVYLQMNSLRAEDTAVYFCARDPNSSDYPYGAYGMGLWGPGTLVTVSS

SEQ ID NO: 6 - protein, 124 aa, Artificial Sequence
  VH
EVQLVESGGGLVQPGGSLRLSCAASGFSLSSSAVSWVRQAPGKGLEWIGAIGSGGTSYYSNWAKSRFTISRDNSKNTLYLQMNSLRAEDTAVYYCARDPNSSDYPYGAYGMGLWGQGTLVTVSS

SEQ ID NO: 7 - protein, 124 aa, Artificial Sequence
  VH
EVQLVESGGGLVQPGGSLRLSCAASGFSLSSSAVSWVRQAPGKGLEWIGAIGSGGTSYYSNWAKSRFTISRDNSKNTLYLQMNSLRAEDTAVYYCARDPNSSDYPYGAYGMGLWGPGTLVTVSS

SEQ ID NO: 8 - protein, 110 aa, Artificial Sequence
  VL
DIQMTQSPSSLSASVGDRVTITCQVSQSISSYLSWYQQKPGKAPKLLIYYASTLASGVPSRFSGSGSGTDFTLTISSLQPEDFATYYCQQGYITSNVGNAFGGGTKVEIK

SEQ ID NO: 9 - protein, 110 aa, Artificial Sequence
  VL
DIQMTQSPSSLSASVGDRVTITCQVSQSISSYLSWYQQKPGKPPKLLIYYASTLASGVPSRFSGSGSGTDFTLTISSLQPEDFATYYCQQGYITSNVGNAFGGGTKVEIK

SEQ ID NO: 10 - protein, 110 aa, Artificial Sequence
  VL
DIQMTQSPSFLSASVGDRVTITCQVSQSISSYLSWYQQKPGKAPKLLIYYASTLASGVPSRFSGSGSGTDFTLTISSLQPEDFATYYCQQGYITSNVGNAFGGGTKVEIK

SEQ ID NO: 11 - protein, 110 aa, Artificial Sequence
  VL
DIQLTQSPSFLSASVGDRVTITCQVSQSISSYLSWYQQKPGKAPKLLIYYASTLASGVPSRFSGSGSGTEFTLTISSLQPEDFATYYCQQGYITSNVGNAFGGGTKVEIK

SEQ ID NO: 12 - protein, 25 aa, Artificial Sequence
  HFR1
EVQLVESGGGLIQPGGSLRLSCAAS

SEQ ID NO: 13 - protein, 8 aa, Artificial Sequence
  HCDR1
GFSLSSSA

SEQ ID NO: 14 - protein, 17 aa, Artificial Sequence
  HFR2
VSWVRQAPGKGLEWIGA

SEQ ID NO: 15 - protein, 7 aa, Artificial Sequence
  HCDR2
IGSGGTS

SEQ ID NO: 16 - protein, 38 aa, Artificial Sequence
  HFR3
YYSNWAKSRFTISRDNSKNTLYLQMNSLRAEDTAVYYC

SEQ ID NO: 17 - protein, 18 aa, Artificial Sequence
  HCDR3
ARDPNSSDYPYGAYGMGL

SEQ ID NO: 18 - protein, 11 aa, Artificial Sequence
  HFR4
WGRGTLVTVSS

SEQ ID NO: 19 - protein, 38 aa, Artificial Sequence
  HFR3
YYSNWAKSRFTISRDNSKNTLYLQMNSLRAEDTAVYFC

SEQ ID NO: 20 - protein, 38 aa, Artificial Sequence
  HFR3
YYSNWAKSRFTISRDTSKNTVYLQMNSLRAEDTAVYFC

SEQ ID NO: 21 - protein, 11 aa, Artificial Sequence
  HFR4
WGPGTLVTVSS

SEQ ID NO: 22 - protein, 25 aa, Artificial Sequence [sequence omitted]

SEQ ID NO: 23 - protein, 11 aa, Artificial Sequence
  HFR4
WGQGTLVTVSS

SEQ ID NO: 24 - protein, 26 aa, Artificial Sequence
  LFR1
DIQMTQSPSSLSASVGDRVTITCQVS

SEQ ID NO: 25 - protein, 6 aa, Artificial Sequence
  LCDR1
QSISSY

SEQ ID NO: 26 - protein, 17 aa, Artificial Sequence
  LFR2
LSWYQQKPGKAPKLLIY

SEQ ID NO: 27 - protein, 3 aa, Artificial Sequence
  LCDR2
YAS

SEQ ID NO: 28 - protein, 36 aa, Artificial Sequence
  LFR3
TLASGVPSRFSGSGSGTDFTLTISSLQPEDFATYYC

SEQ ID NO: 29 - protein, 12 aa, Artificial Sequence
  LCDR3
QQGYITSNVGNA

SEQ ID NO: 30 - protein, 10 aa, Artificial Sequence
  LFR4
FGGGTKVEIK

SEQ ID NO: 31 - protein, 17 aa, Artificial Sequence
  LFR2
LSWYQQKPGKPPKLLIY

SEQ ID NO: 32 - protein, 26 aa, Artificial Sequence
  LFR1
DIQMTQSPSFLSASVGDRVTITCQVS

SEQ ID NO: 33 - protein, 26 aa, Artificial Sequence
  LFR1
DIQLTQSPSFLSASVGDRVTITCQVS

SEQ ID NO: 34 - protein, 36 aa, Artificial Sequence
  LFR3
TLASGVPSRFSGSGSGTEFTLTISSLQPEDFATYYC

SEQ ID NO: 35 - protein, 25 aa, Artificial Sequence
  HFR1
QQSVKESEGGLFKPTDTLTLTCTVS

SEQ ID NO: 36 - protein, 17 aa, Artificial Sequence
  HFR2
VSWVRQAPGNGLEWIGA

SEQ ID NO: 37 - protein, 38 aa, Artificial Sequence
  HFR3
YYSNWAKSRSTITRDTNLNTVTLKMTSLTAADTATYFC

SEQ ID NO: 38 - protein, 27 aa, Artificial Sequence
  LFR1
AELDLTQTPASVEVAAGGTVTINCQVS

SEQ ID NO: 39 - protein, 17 aa, Artificial Sequence
  LFR2
LSWYQQKPGQPPKLLIY

SEQ ID NO: 40 - protein, 36 aa, Artificial Sequence
  LFR3
TLASGVPSRFSGSGSGTEFTLTISGVQCDDAATYYC

SEQ ID NO: 41 - protein, 10 aa, Artificial Sequence
  LFR4
FGGGTEVVVK

SEQ ID NO: 42 - protein, 25 aa, Artificial Sequence
  HFR1
  misc_feature (12)..(12): Xaa = Phe, Ile, Leu, Met, Val, Trp or Tyr
EVQLVESGGGLXQPGGSLRLSCAAS

SEQ ID NO: 43 - protein, 38 aa, Artificial Sequence
  HFR3
  misc_feature (16)..(16): Xaa = Asp, Glu, His, Lys, Asn, Gln, Arg, Ser or Thr
  misc_feature (21)..(21): Xaa = Phe, Ile, Leu, Met, Val, Trp or Tyr
  misc_feature (37)..(37): Xaa = Phe, His, Ile, Leu, Met, Val, Trp or Tyr
YYSNWAKSRFTISRDXSKNTXYLQMNSLRAEDTAVYXC

SEQ ID NO: 44 - protein, 11 aa, Artificial Sequence
  HFR4
  misc_feature (3)..(3): Xaa = Pro, Gln or Arg
WGXGTLVTVSS

SEQ ID NO: 45 - protein, 26 aa, Artificial Sequence
  LFR1
  misc_feature (4)..(4): Xaa = Phe, Ile, Leu, Met, Val, Trp or Tyr
  misc_feature (10)..(10): Xaa = Phe or Ser
DIQXTQSPSXLSASVGDRVTITCQVS

SEQ ID NO: 46 - protein, 17 aa, Artificial Sequence
  LFR2
  misc_feature (11)..(11): Xaa = Ala, Gly, Pro or Ser
LSWYQQKPGKXPKLLIY

SEQ ID NO: 47 - protein, 36 aa, Artificial Sequence
  LFR3
  misc_feature (18)..(18): Xaa = Asp, Glu, His, Lys, Asn, Gln, Arg, Ser or Thr
TLASGVPSRFSGSGSGTXFTLTISSLQPEDFATYYC

SEQ ID NO: 48 - protein, 330 aa, Artificial Sequence [sequence omitted]

SEQ ID NO: 49 - protein, 107 aa, Artificial Sequence [sequence omitted]